(19) United States
(12) Patent Application Publication
(10) Pub. No.: US 2026/0083410 A1
(43) Pub. Date: Mar. 26, 2026

(54) VOICE INTERROGATION, CONTROL, AND AUTHENTICATION OF CONNECTED DRUG DELIVERY DEVICES

(71) Applicant: SHL Medical AG, Zug (CH)

(72) Inventors: Martin Michael Coyne, III, Boonton, NJ (US); Katherine Ford Brigham, Boonton, NJ (US); Christopher James Franzese, Boonton, NJ (US)

(21) Appl. No.: 19/109,340

(22) PCT Filed: Sep. 1, 2023
(86) PCT No.: PCT/EP2023/074083

§ 371 Date: Mar. 6, 2025

Related U.S. Application Data
(60) Provisional application No. 63/405,013, filed on Sep. 9, 2022.

(30) Foreign Application Priority Data
Feb. 27, 2023 (EP) 23158687.6

(51) Int. Cl. A61B 5/00; G10L 17/06; G10L 17/24; G10L 25/66; G16H 10/60

(52) U.S. Cl. CPC A61B 5/749; A61B 5/4803; A61B 5/4839; A61B 5/4848; A61B 5/741; A61B 5/747; G10L 17/06; G10L 17/24; G10L 25/66; G16H 10/60

(57) ABSTRACT

The present disclosure relates to systems and methods for using functionalities of voice activated assistant systems to monitor and control delivery of therapeutic medicines. An individual makes spoken requests to a voice assistant provisioned with a software skill coupled to a drug delivery device. Spoken requests directed to the system may interrogate various aspects of medication, medication delivery, or status of a patient receiving medication with the device. Other spoken requests directed to the system may control various aspects of drug delivery. Still other spoken requests directed to the system may enable emergency response to a patient using the device, as in the case of an acute systemic infusion reaction to one or more medicines contained within the drug delivery device. Systems and methods disclosed herein enable restriction of certain requests and commands to an authorized user of the device, provide methods to distinguish authorized from unauthorized users before responding to a user request, to prevent inadvertent interrogation and/or control of the apparatus, and to avoid verbalization of private patient information.

### TECHNICAL FIELD

Principles and embodiments of the present disclosure relate to systems and methods for using functionalities of voice assistant systems to improve monitoring and delivery of therapeutic medicines. More particularly, the present disclosure is directed towards more intuitive approaches to monitor and control drug delivery systems, especially those delivering more complicated medication regimens or medications that may cause a patient to suffer a reaction, such as a systemic infusion reaction.

### BACKGROUND

Patients often require complex, ongoing treatment to manage their conditions. Such treatments may include individual medications or multi-medication regimens given by one or more physiologic routes of administration. Medications, medication regimens, and routes of administration generally correspond to a specific disease state and treatment regimen and may be customized to a specific patient's clinical needs, disease progression, or physical and/or cognitive limitations. Individual therapies and medication regimens are administered using a variety of drug delivery systems, which may be familiar to clinicians in an inpatient setting but may be overwhelming for patients at home who lack clinical training, have low literacy or health literacy, or suffer limitations from underlying disease.

Many drug delivery devices used to deliver treatments are disposable, single-use devices that are mass-produced. Practical cost and manufacturing constraints limit inclusion of advanced functionality that would improve intuitiveness and patient usability. As a result, many drug delivery devices are limited to simple interaction elements including pushbuttons, mechanical audible/tactile feedback mechanisms, or moving visual indicators. There is a need for improved methods of feedback and more intuitive user interactions that would be prohibitive to implement on lower-cost, disposable devices themselves. These needs are particularly relevant for delivery of more complex therapies, such as multi-medication regimens, especially when self-administered at home by a patient or user lacking clinical training, such as a caregiver, family member, or friend.

Additionally, certain medications have a higher relative propensity to induce sudden, unpredictable systemic reactions related to the origin, pharmaceutics, or mode of action of the medication upon the body. While rare, systemic reactions are potentially fatal, demanding immediate halt to medication administration, and emergency administration of one or more counteracting medications. Clinicians are skilled at identifying and responding to systemic reactions. However, in the home setting in the absence of a trained clinician, patients must rely on themselves, a caregiver (e.g., family member), or emergency responders for intervention. As such, there is a need for drug delivery devices that enable safe home use by patients themselves or lay (non-clinical) caregivers, enabling effective emergency treatment if needed.

Voice assistant systems (also called “voice-activated assistants,” “voice assistant devices,” or “smart speakers”) are increasingly commonplace in homes. Such colloquial terms, used herein as terms of convenience, generally refer to devices with hardware and software configured to listen for and be activated by a wakeword spoken by a user of the device, to capture a spoken request by the user after the wakeword, to interpret the spoken request by the user, to determine the content and intent of the request, and to formulate and provide a response to a requester as computer-generated natural spoken language.

Voice assistant systems may be implemented in with purpose-built hardware and/or software (e.g., proprietary platforms or an open-source platform, such as Mycroft. AI) or in a multi-purpose hardware and/or software (e.g., wireless speakers, a smartphone/tablet, a smart watch, a desktop computer, or a laptop). Voice assistant systems may be uni-modal (i.e., receive requests, and respond using only voice interaction) or multi-modal (i.e., receive verbal requests, and respond using voice and visual interaction).

Voice assistant systems are intentionally extensible and through installation of additional software-based known as “skills” or “actions.” Skills, used herein as a term of convenience, refers to software modules or programs that can be installed and enabled on a voice assistant system's component hardware and/or software to provide additional features or functionality. Skills broaden the range of potential requests and responses that a voice assistant system can accommodate, and are the primary means by which a voice assistant system is tailored to the needs and priorities of each user.

### SUMMARY

The present disclosure provides new and/or improved apparatuses, configurations, systems, and methods for interacting with and/or controlling a drug delivery system, the system comprising a drug delivery device to deliver medication coupled to a voice assistant service configured to provide natural language input and output, the voice assistant also being configured with an associated computer program or “skill” coupling the voice assistant service and drug delivery device.

The integration of drug delivery devices with voice assistant systems enables natural, conversational interactions, to allow complex topics of medications, regimen, device usage, and administration to be more easily understood. Complexity of the actual drug delivery device is also reduced, as the processing of user requests and responses is handled by software already provided in the voice assistant system, augmented by a voice assistant software or “skill” coupled to the drug delivery device. The apparatus and methods described herein are particularly advantageous for users lacking clinical training (such as the patient themselves or a lay caregiver or family member), those with cognitive or physical limitations, or for complex therapies.

The apparatus enables a user to direct spoken queries and/or commands to the voice assistant component of the apparatus before, during, and after administration of medications. The voice assistant service configured within the apparatus is provided with hardware and/or software to listen for a predetermined wakeword to activate the voice assistant, to recognize a user's spoken request following the wakeword and related to the drug delivery device, to receive and record the spoken request, to parse the recorded request for intent and context, and to activate a voice assistant skill configured to interface with the coupled drug delivery device and receive from the voice assistant the parsed intent and/or context of a user request related to the delivery device. The voice assistant skill may also be configured to query or control one or more aspects of the coupled drug delivery device corresponding to the parsed intent and/or context of the user request, to compose a relevant response based on the status of the coupled drug delivery device or patient provided the coupled delivery device, and to provide data to the voice assistant service to generate computer-generated spoken response in natural language to the requester-user.

In one or more embodiments, a response generated by the drug delivery device skill comprises information on one or more aspects of medication delivery and/or patient status. In one or more embodiments of the apparatus, a response generated by the drug delivery device skill comprises an instruction to the coupled drug delivery device to control one or more aspects of medication delivery to the patient. In one or more embodiments of the apparatus, the response generated by the drug delivery device skill comprises one or more instructional and/or informational aspects related to emergency treatment of a patient using the drug delivery system.

In some embodiments, a response generated by the apparatus includes one or more data elements provided by one or more sensors contained within the drug delivery device. In some embodiments, a response generated by the apparatus includes one or more data elements provided by one or more sensors on, worn, or in proximity to a patient receiving medications using the drug delivery system. In some embodiments, a response generated by the apparatus includes one or more data elements contained in an electronic health record associated with a patient receiving medications using the drug delivery system.

The response is provided by the drug delivery device skill to the voice assistant system, which presents the response to the requester in natural language as audible, spoken speech. In some embodiments, the voice assistant system supplements the audible, spoken response with visual information also provided by the drug delivery skill.

In one or more embodiments, a user of the apparatus is the patient receiving medication with the drug delivery device. In one or more embodiments, a user of the apparatus is a person providing one or more aspects of care or supervision for the patient, including by way of example but not limitation, a nonclinical caregiver, family member, aide, hospice worker, nurse, pharmacist, doctor, respiratory therapist, or emergency responder.

In some embodiments, the system is configured to prevent inadvertent or unauthorized interrogation or control of the system. In some embodiments, the system is configured to establish and verify authorized user(s) that may make verbal requests of the apparatus, to limit certain requests to authorized users, and/or to permit certain requests by unauthorized users. In some embodiments, the system is configured to protect against unintended and potentially hazardous operation by inadvertent mimicry of spoken commands, as by a child or household pet. In some embodiments, the system is configured to prevent inadvertent disclosure of a patient's private information as part of a verbalized response by the apparatus.

These as well as other aspects, advantages, and alternatives, will become apparent to those of ordinary skill in the art by reading the following detailed description, with reference where appropriate to the accompanying drawings.

### BRIEF DESCRIPTION OF THE FIGURES

A clear understanding of the key features of the invention summarized above may be had by reference to the appended drawings, which illustrate the method and system of the invention, although it will be understood that such drawings depict various preferred and alternative embodiments of the invention and, therefore, are not to be considered as limiting its scope regarding other embodiments enabled by the invention

FIG. 1 shows an exemplary configuration of various input, output, processing, and data storage components of a generic voice assistant system and interactions thereof, according to an example embodiment.

FIG. 2 shows an exemplary configuration of a drug delivery system, controller, coupled sensors, and voice assistant system, according to an example embodiment.

FIG. 3 presents in tabular format exemplary requests and responses related to medication administration status for a patient receiving medication using a coupled drug delivery system and voice assistant system, according to an example embodiment.

FIG. 4 presents in tabular format exemplary requests and responses related to the current physiologic status of a patient receiving medication using a coupled drug delivery system and voice assistant system, according to an example embodiment.

FIG. 5 presents in tabular format exemplary requests and responses related to the current delivery device configuration and/or using a coupled drug delivery system and voice assistant system, according to an example embodiment.

FIGS. 6A-6D presents in tabular format exemplary requests and responses related to emergency response and/or intervention for a patient receiving medication using a coupled drug delivery system and voice assistant system, according to an example embodiment.

FIGS. 7A-7B presents in tabular format exemplary requests and responses related to patient assessment using one or more patient reported outcomes or clinical assessments using a coupled drug delivery system and voice assistant system, according to an example embodiment.

FIG. 8 shows a schematic drawing of components of coupled drug delivery system and voice assistant system, according to an example embodiment.

FIG. 9 shows a schematic drawing of a process to evaluate a request made by a user of the system, configured to distinguish between commands requiring authorization and those that do not and to establish an authorized user of the coupled drug delivery system and voice assistant system, according to an example embodiment.

FIGS. 10A-10G presents in tabular format exemplary requests and responses to control a drug delivery device and coupled drug delivery system and voice assistant system, according to an example embodiment.

FIGS. 11A-11C shows a schematic drawing of a process to evaluate a request made by a user of the system, configured to distinguish between interrogation commands and control commands, according to an example embodiment.

### DETAILED DESCRIPTION

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an example embodiment may include elements that are not illustrated in the Figures.

In the Figures, the blocks may represent operations and/or portions thereof and lines connecting the various blocks do not imply any particular order or dependency of the operations or portions thereof. It will be understood that not all dependencies among the various disclosed operations are necessarily represented. Any of the Figures and the accompanying disclosure describing the operations of the method(s) set forth herein should not be interpreted as necessarily determining a sequence in which the operations are to be performed. Rather, although one illustrative order is indicated, it is to be understood that the sequence of the operations may be modified when appropriate. Accordingly, certain operations may be performed in a different order or simultaneously. Additionally, those skilled in the art will appreciate that not all operations described need be performed.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

Reference herein to “one embodiment” or “one example” means that one or more feature, structure, or characteristic described in connection with the example is included in at least one implementation. The phrases “one embodiment” or “one example” in various places in the specification may or may not be referring to the same example.

As used herein, apparatus, element and method “configured to” perform a specified function is indeed capable of performing the specified function without any alteration, rather than merely having potential to perform the specified function after further modification. In other words, the apparatus, element, and method “configured to” perform a specified function is specifically selected, created, implemented, utilized, programmed, and/or designed for the purpose of performing the specified function. As used herein, “configured to” refers to existing characteristics of an apparatus, element, and method which enable the apparatus, element, and method to perform the specified function without further modification. For purposes of this disclosure, an apparatus, element, and method described as being “configured to” perform a particular function can additionally or alternatively be described as being “adapted to” and/or as being “operative to”perform that function.

The present disclosure advances the art by providing embodiments to enable patients, caregivers, healthcare providers, and emergency personnel to interact with and/or control a drug delivery device indirectly via a voice assistant in a variety of ways. As will be appreciated by one skilled in the art, there are numerous ways of carrying out the examples, improvements and arrangements of systems and methods disclosed herein. Although reference will be made to the exemplary embodiments depicted in the drawings and the following descriptions, the embodiments disclosed herein are not meant to be exhaustive of the various alternative designs and embodiments that are encompassed by the present disclosure.

### A. Introduction to Voice Assistants

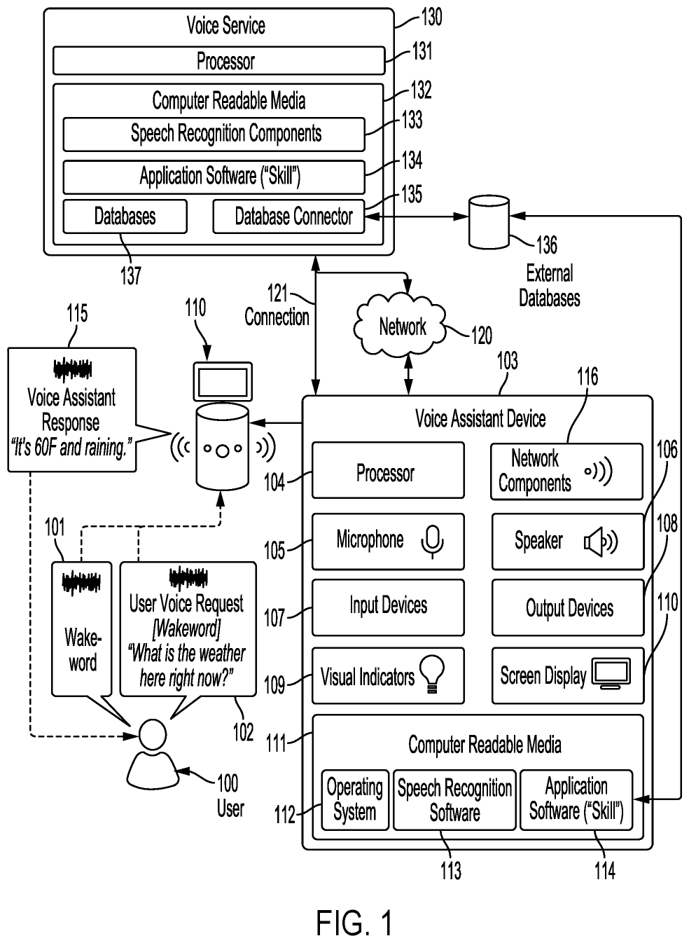

Referring to FIG. 1, a voice assistant system generally includes a voice assistant device 103 comprising a processor 104, microphone 105, speaker 106, network access components 116 (such as a wireless unit and antenna), input devices 107, output devices 108, visual indicators 109, an optional visual display screen 110, and computer-readable media 111 comprising an operating system 112, local speech recognition software 113, and one or more application software or skills 114. The computer-readable media 111 stores computer-readable instructions that, when executed by the processor 104, cause the processor 104 to perform functions. Voice assistant device 103 is configured to access, either through direct connection 121 or via the network access components 116 and/or a network 120, a voice service 130. Voice service 130 comprises a processor 131, computer readable media 132 with speech recognition components 133, application software 134, an optional internal database 137, and one or more optional external database connections 135 to one or more external databases 136. External databases 136 are available to the voice assistant system, but do not comprise part of the voice service 130.

Application software 114 and remote application software 134 collectively comprise what is colloquially known as a “skill,” a term of convenience. As used herein, “skills” refer to software applications that are either installed on or provided within a voice assistant system and expand the functionality provided by a voice assistant system. In one or more embodiments, either or both of application software 114 and application software 134 collectively rely on speech recognition software 113 and speech recognition components 133 provided by a voice assistant device 103 and voice service 130 respectively. In one or more embodiments, either or both of application software 114 and application software 134 perform one or more functions that are not performed by the voice assistant natively in the absence of the installed skill. In one or more embodiments, either or both of application software 114 and application software 134 may be configured to interact with one or more software or hardware components of the voice assistant device 103 or voice service 130 to provide additional functionality to a voice assistant system. Thousands of skills exist spanning many topics, such as fitness, weather, music, smart home controls, home security, and cooking. The present disclosure is partially directed to a skill by which a voice assistant system may be interfaced to interrogate the status of and/or control a drug delivery device as described herein.

Utterance of a predefined wakeword 101 by a user 100 in proximity to voice assistant device 103 activates the voice assistant system to identify and record a voice request 102 spoken by a user 100 in proximity to the voice assistant device 103 using speech recognition software 113, then communicating it to a voice service 130 via a direct connection 121 or a network access component 116 and network 120. Voice service 130 processes request 102 using speech recognition components 133, including software instructions such as automatic speech recognition, natural language processing, or text-to-speech algorithms. Voice service 130 may also engage either or both of application software 114 or 134 to formulate a response 115, which is delivered to the user 100 audibly using one or more of the speaker 106, visual display screen 110, visual indicators 109, or other output devices 108. In an alternative embodiment, the visual display screen 110 may be separate from the voice assistant device 103. In an alternative embodiment, the visual display screen 110 and/or speaker 106 may comprise a casting device (e.g., a smart television, phone, or tablet) to support delivery of additional content or deliver responses. In one or more embodiments, the visual display screen 110 may display additional information not contained in the audible response provided to the user by the speaker 106.

In formulating a response 115, the system and associated application software 114 and/or 134 may also rely upon databases 136 and 137 containing relevant data. Database 136 may be accessible to the voice service 130, voice assistant device 103, or skill 114. External database 137 is accessible to voice service 130 but is not part of the voice assistant system and may be used for other purposes. For instance, external database 137 may comprise one or more data related to the drug delivery system, one or more medications, or one or more aspects of a patient using the drug delivery system, all as described herein. External database 137 may also comprise information contained in an electronic health record system, including patient identifiers (e.g., name, address, medical record number), patient demographic information (e.g., age, race, gender), healthcare visit history (e.g., hospital admissions, outpatient visits), patient medical history, flowchart values (e.g., height and weight, vital signs, pain assessment, oxygen therapy, invasive monitors), patient lines, drains, and airway information, laboratory values (e.g., chemistry, hematology, coagulation, microbiology), radiology findings, medication history (e.g., active prescriptions, dispensing history, administration parameters, adverse events, infusion reactions, etc.), electronically entered or scanned provider notes, active or signed and held orders (e.g., treatment plans, therapy plans, order sets, laboratory orders, diet orders, contingent medication administrations, nursing or other provider communications), and the medication administration record. One or more data in external database 137 may also be replicated in database 136. Information in external database 137 may thus be accessible to many different systems, including voice service 130 and skill 114. Information in database 136 may be accessible to the voice service 130 and skill 114, or may be made available to other services aside from the present invention.

### B. Drug Delivery “skill”

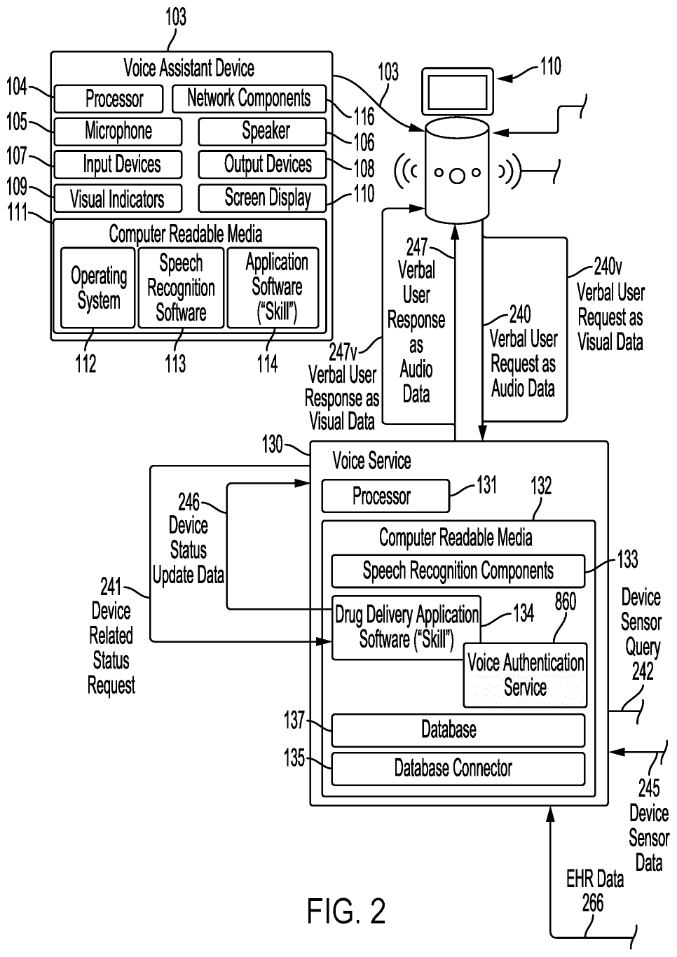

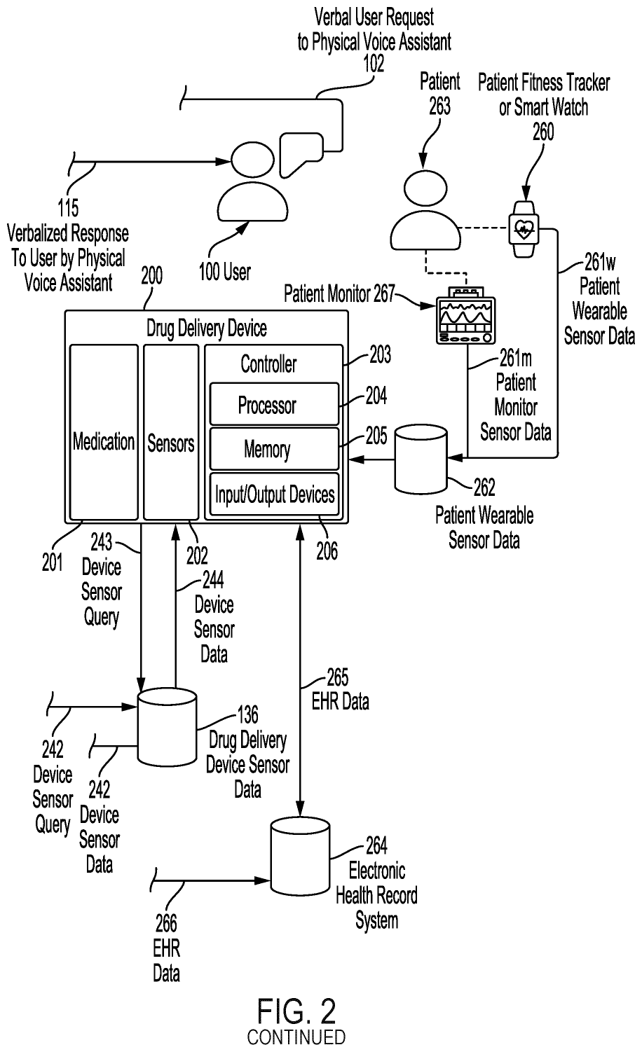

FIG. 2 illustrates an exemplary configuration of components for a drug delivery system enabled to work with a voice assistant system in accordance with the present invention. In one or more embodiments, a drug delivery system 200 administers one or more medications 201 and is provided with a controller 203 comprising a processor 204 and sensors 202, a memory 205, and input/output devices 206. Said processor 204, sensors 202, a memory 205, and input/output devices 206 are coupled to the processor 204 and are provided support circuits to provide communication between the different components of the system, namely the components of the system described herein. Sensors 202 provide data from the drug delivery device 200, which are communicated via the controller 203 and coupled input/output devices 206 to an external database 136. In one or more embodiments, the sensor 202 data communicated via the controller 203 and coupled input/output devices 206 to an external database 136 includes one or more parameters related to drug delivery device or patient physiologic status. In one or more embodiments, a drug delivery device skill 134 is installed on a physical voice assistant device 103 and/or a voice service 130 associated with the voice assistant system.

Medication 201 within drug delivery device 200 is shown singly in FIG. 2 by way of illustration but not limitation. Medication 201 within drug delivery device 200 may be a single medication (e.g., pertuzumab), a combination therapy of two separate medications (e.g., medication regimen, such as pertuzumab and trastuzumab), a co-formulation of two or more medications (e.g., immunoglobulin and hyaluronidase), or any combination thereto. Medication 201 may be delivered by any combination of one or more routes of administration, including parenteral, enteral, intrathecal, epidural, percutaneous, transdermal, topical, intravenous, intraosseous, intravitreal, or pulmonary.

In one or more embodiments, voice service 130 may be provided in one or more of a physical voice assistant device 103, in the drug delivery system 200, in a companion application (e.g., a mobile phone application) designed to work in conjunction with drug delivery system 200, in a smart television or other household electronic device, or other devices suitable for voice interactions.

A user voice request 102 is made by a user 100 in proximity to physical voice assistant device 103, which identifies and records said voice request. Voice assistant device 103 converts the user voice request 102 into a verbal user request as audio data 240, which is communicated to a voice service 130. Voice service 130 then additionally processes said audio data 240 using speech recognition software components to identify the nature of the request. In or more embodiments, audio data 240 is supplemented with visual data 240v. If the request regards the drug delivery system 200, the voice service 130 provides the drug delivery skill 134 with a device related request as data 241 and awaits a response from the skill 134. To formulate a response to the request 241, drug delivery skill 134 makes one or more sensor queries 242 of an external database 136 containing data from one or more sensors 202 contained within the drug delivery device 200. Alternatively, sensor query 242 may trigger a device sensor query 243 to obtain updated device sensor data 244 from sensors 202 contained within the drug delivery device 200. In either case, the external database 136 returns sensor data 245 to the drug delivery skill 134 for processing, returning a device status response as data 246 to voice service 130.

In one or more embodiments, voice service 130 converts the device status response as data 246 into verbal user response as audio data 247, which is delivered to the user 100 as natural language speech by the speaker 106 contained within the voice assistant device 103. In or more embodiments, audio data 247 is supplemented with visual data 247v presented to the user 100 on a display screen 110.

Referring to FIG. 2, requests 102 are made to voice assistant device 103 by a user 100, and responses 115 are provided to a user 100 of the apparatus by the voice assistant device 103. Requests 102 and responses 115 comprise a request-response pair related to one or more aspects of the drug delivery device and/or a patient using the drug delivery device. As used herein, “request-response pair” refers to two way interactions between a voice assistant a user of the voice assistant. Requests may be initiated by a user to the voice assistant, and vice-versa. Requests initiated by the user result in a response from the voice assistant. Requests initiated by the voice assistant result in a response from the user. Responses may optionally include a follow-on request, as for clarification, confirmation, or other purposes based on the context of the request or other factors.

In one or more embodiments, a user of the device may issue requests and receive responses before, during, and/or after administration with the drug delivery device 200. For instance, requests may be issued and responses received while the drug delivery device 200 remains packaged (e.g., from the manufacturer), when the drug delivery device 200 is ready for use but not yet in use, while the drug delivery device 200 is attached to a patient, while the drug delivery device 200 is unattached from a patient after use, while the drug delivery device 200 is disposed of, and after disposal of the drug delivery device 200. These different aspects may be combined or selected based on the drug delivery device, medications, therapeutic regimen, patient circumstances, medication or patient safety, or other factors.

In one or more embodiments, said request 102 may be any question, inquiry, interrogation, instruction, command, statement, or phrase related to one or more aspects of the function, readiness, operation, progress, error states, configuration, or misconfiguration related to the drug delivery system and/or the drug delivery device 200. For instance, said request 102 may be a question, inquiry, interrogation, instruction, command, statement, or phrase related to one or more aspects of one or more medications delivered to a patient 263 by the drug delivery device 200. Alternatively, request 102 may be any question, inquiry, interrogation, instruction, command, statement, or phrase related to one or more aspects of a patient 263 using the drug delivery system and/or the drug delivery device 200, such as one or more physiologic parameters, a patient self-report of wellness, symptoms, or perceived condition, or data or results from a clinical assessment or patient-reported outcome (also known as “PRO” or “ePRO”).

A request 102 may result in a response 115 from the system to a user 100 as described or may result in no response from the system to a user 100. A request 102 may also result in a response 115 that notifies a third party, such as a caregiver, relative, or clinician.

A user 100 making a request 102 of the apparatus may be any person, for instance, an adult, a child, or an adolescent. A user 100 making a request 102 of the apparatus may also be the patient receiving a medication with a drug delivery system 200 in proximity to where the voice assistant device 103 is located. A user 100 making a request 102 of the apparatus may also be a caregiver, healthcare provider, or emergency responder to the patient. Moreover, user 100 need not have prior knowledge (e.g., training on) of the delivery device 200 used with voice assistant device 103.

In one or more embodiments, the apparatus is configured to distinguish between an authorized and non-authorized user of the device. In one or more embodiments, the apparatus is configured to distinguish between an authorized and non-authorized user of the device based on the content, nature, or intent of the request. In one or more embodiments, the apparatus is configured to allow or disallow certain requests 102 based on a user being authorized or unauthorized. For instance, a first user 100 may not be authorized to make requests 102 to the voice assistant to control the delivery device 200, but may be authorized to inquire as to the delivery device 200 status and/or patient 263 status. In contrast, a second user 100 may be authorized to make requests 102 to the voice assistant to both inquire as to the delivery device 200 status and/or patient 263 status and to control the delivery device 200. Additionally, the apparatus may be configured to preclude requests 102 from a person, animal, or system capable of mimicking human speech or sounds (e.g., a parrot, cat, or voice recorder) that may cause unintended or unsafe operation of the apparatus.

In one or more embodiments, the voice assistant device 103 is activated by a user's 100 utterance of a wakeword 101. The wakeword may be uttered in isolation or may be immediately followed by a request 102. In one or more embodiments, the wakeword is a specific word, phrase, or invocation specific to the voice assistant device 103 platform used. By way of example, this could include “Hey Siri,” “Hey Google,” or “Alexa” for Apple Home Kit, Google Nest, and Amazon Alexa voice assistant devices, respectively. Other voice assistant devices may feature different wakewords, allow a customized wakeword, use a wakephrase consisting of several words, or may allow each user to select a specific individual wakeword. In one or more embodiments, the wakeword 101 activates the voice assistant and drug delivery skill explicitly (“Hey Google, Talk to <drug delivery skill>”). In one or more embodiments, the wakeword 101 activates the voice assistant and drug delivery skill explicitly (“Hey Google, Talk to <drug delivery skill>”), which then instructs the voice assistant device 103 to activate the drug delivery skill 134 and verbally prompt the user 100 for their request related to the drug delivery device (“What would you like help with? You can ask me ‘How much time's left in my injection’ or ‘What medication am I getting right now?’”). In one or more embodiments, the wakeword 101 and request 102 activates the voice assistant and drug delivery skill implicitly through the context of the request 102 (“Hey Google, how much more time to go on my nebulizer?”)

In one or more embodiments, instructions related to the wakeword 101, voice assistant device 103 activation, and/or drug delivery skill 134 are provided on the drug delivery device 200, voice assistant device 103, or one or both of the packaging materials for the apparatus or components. Instructions related to use of the apparatus may also be provided on visual display 110 before, during and/or after administration of medication.

In one or more embodiments, the drug delivery skill 134 is installed on the voice assistant system device 103 or voice service 130 by a user, thereby making it available for use. In one or more embodiments, the drug delivery skill 134 may be pre-installed on the voice assistant device 103 or voice service 130 without intervention or added steps. In one or more embodiments, the drug delivery skill 134 may be pre-installed on the voice assistant system 103 or 130 and provided to a user of the drug delivery device at initiation of therapy (e.g., as part of a starter or “Welcome” kit). In an alternative embodiment, the voice enabled assistant 103 or 130 and associated controlling computer program and/or skill 134 is installed in a commercially available unit (e.g. Amazon Alexa, Apple Homekit, or Google Nest). In an alternative embodiment, the voice enabled assistant 103 or 130 and one or more aspects of the associated controlling computer program and/or skill 134 is installed on a mobile phone, tablet, or smart watch. In an alternative embodiment, the voice enabled assistant 103 or 130 and/or one or more aspects of the associated controlling computer program and/or skill 134 is installed on the drug delivery device itself.

In one or more embodiments, the drug delivery skill 134 is configured to formulate a spoken response 246, 247 related to one or more aspects of the drug delivery device 200 and/or a patient using the drug delivery device 211. In one or more embodiments, the drug delivery skill 134 is configured to formulate a multi-modal response comprising a verbal component 246, 247 and a visual component 247v, both components related to one or more aspects of the drug delivery device 200 and/or a patient using the drug delivery device 211. In one or more embodiments, the drug delivery skill 134 is configured to notify one or more of emergency responders, a patient's designated emergency contact, or a patient's healthcare provider. In one or more embodiments, the notification provided by the drug delivery skill 131 comprises one or more of an electronic notification, SMS/text message, application “push” notification (e.g., within a mobile phone application), by electronic health record messaging system, telephone call, or visual display on a smart TV, as non-limiting examples.

In one or more embodiments, sensor data 244 communicated by controller 203 to external database 136 and/or drug delivery skill 134 comprises one or more parameters associated with medication administration, an aspect of patient physiologic status, or an aspect of systemic infusion reaction to an administered medication and/or medication regimen.

For example, parameters associated with medication administration may include skin contact with the patient interface, skin temperature, current medication under administration, administration start time, administration end time, administration rate, reservoir status, pre-medications administered, post-medications administered, emergency medications administered, administration time zone, medication order identifier, administration geolocation data, and patient identifier. An aspect of patient physiologic status may include, for example, heart rate, blood pressure, respiratory rate, blood oxygen saturation (SpO2), and body temperature.

An aspect of systemic infusion reaction may be related to anaphylaxis or anaphylactoid reactions, cytokine release syndrome, or tumor lysis syndrome are selected from the group of leukocytosis, leukopenia, neutropenia, thrombocytopenia, abnormalities in liver function (e.g., aminotransferases, bilirubin, international normalized ratio) abnormalities in kidney function (e.g., serum creatinine, estimated glomerular filtration rate), electrolyte abnormalities, hyperuricemia, C-reactive protein, ferritin, or inflammatory cytokines (e.g., interferon gamma, inkerleukin-6).

In one or more embodiments, the system described herein may also utilize sensor data 261w from a patient's smart watch or wearable fitness tracker 260 which may be stored in a second external database 262 and used to formulate a response to a user of the apparatus. In one or more embodiments, external database 136 and second external database 262 comprise either or both of shared and separate databases. In one or more embodiments, sensor query 242 may return sensor data 245 to the drug delivery device skill 134 from either or both of external databases 136 and 262, or other suitable external database. In one or more embodiments, sensor data 261w, 261m from a patient's smart watch, wearable fitness tracker 260, or patient monitor 267 comprise physiologic data regarding an aspect of a patient receiving medication with the apparatus. In one or more embodiments, sensor data 261w, 261m comprise, for example, one or more parameters related to patient physiologic status selected from the group of heart rate, blood pressure, respiratory rate, blood oxygen saturation (SpO2), and body temperature, or alternatively, measures of emotional well-being or emotional sentiment.

In one or more embodiments, the system described herein may also utilize data 265, 266 from an electronic health record system (EHR) 264 containing data for the patient receiving medication with the apparatus. In one or more embodiments, the apparatus is configured to communicate data from the EHR system 264 to one or more of the drug delivery skill 134, the drug delivery device 200, or a database 136 accessible to drug delivery device 200. In one or more embodiments, the apparatus is configured to communicate data from one or more of the drug delivery skill 134, the drug delivery device 200, or a database 136 accessible to drug delivery device 200 to the EHR system 264.

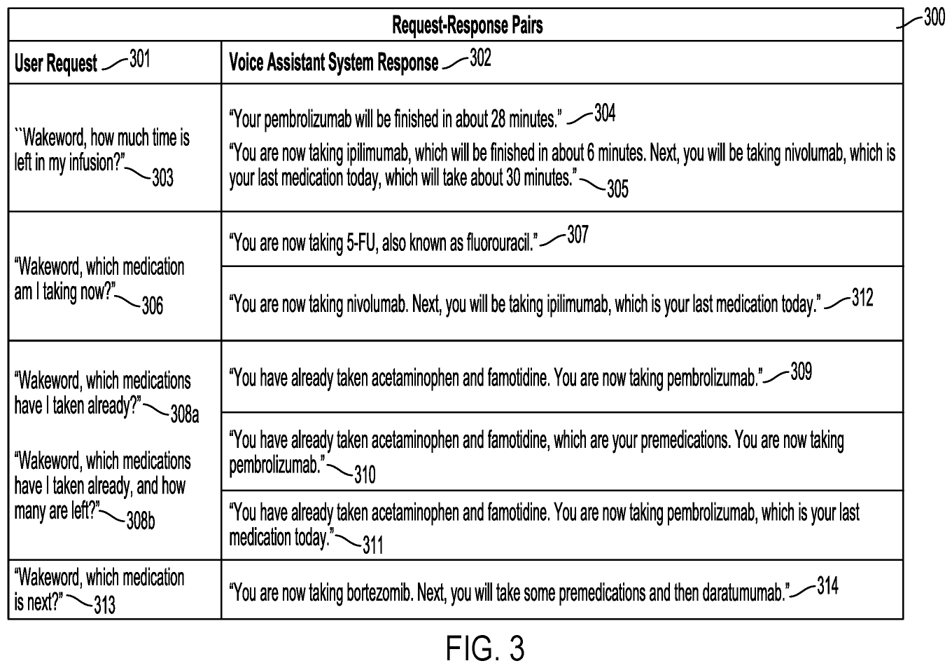

In one or more embodiments, a user of the device may issue requests and receive responses, denoted herein as a request-response pair, a term of convenience. Exemplary illustrative request-response pairs 300, 400, 500, 600, 700, 1000 as shown in FIGS. 3-7 and 10 explain the various circumstances and interactions by which the invention herein may be implemented and used. However, neither the request-response pairs detailed in FIGS. 3-7 and 10 nor descriptions of same herein should be construed as limiting the disclosure herein to those specific request use cases, users, situations, topics, content, phrasing, medications, medication regimens, emergency situations, or routes of administration; those skilled in the art will readily identify many potentially useful responses relevant to the medication delivery process enabled by the disclosure herein. Similarly, grammatic structure in a specific request-response pair shall not be construed as limiting; for instance, a request written in first-person terms may also be restated in third-person terms, or vice versa. Moreover, any request-response pairs, including exemplary illustrative request-response pairs 300, 400, 500, 600, 700, 1000, may be supplemented with a visual response presented on the visual display of a voice assistant (or visual display coupled to a voice assistant) as described herein. A series of illustrative request-response pairs corresponding to different use cases for the apparatus will now be detailed in turn.

### C. Illustrative Use Cases—Interrogating Medication Delivery

In some embodiments, the apparatus may be provisioned to allow interrogation of the voice assistant as to one or more aspects of medications delivered, being delivered, or to be delivered by the drug delivery system. FIG. 3 shows a series of exemplary illustrative pairs 300 of requests 301 and corresponding responses 302 related to the device's medication administration status in accordance with the embodiments described herein. In some embodiments, a user may interrogate the voice-enabled assistant as to the device's medication administration status exemplified in requests 303, 306, 308a, 308b, and 318. Such a request may include, for example, the time remaining in an administration 303, the name of a medication being administered 306, which medications have been taken 308a, which medication is to be administered next 313, or which medications remain 308b. Other aspects of medication delivery will be apparent to those skilled in the art based on the example requests 301, to which other responses may be formulated other than those exemplary responses 302 herein.

An exemplary verbalized response may comprise a recitation of one or more medications being currently administered by the system 304, 305, medications already taken 308, or medications remaining to be administered 309, 310, 311, 312, 314. Some responses may include an estimated time to complete administration of a specific medication 304, 305, or an overall estimated time remaining to complete administration of the full sequence (i.e., regimen) of medications. Alternatively, an exemplary verbalized response may also comprise a recitation of all or part of a medication regimen 309, 310, 311, 312, 314 optionally including the current medication being administered and which other medications remain to be administered. A response may optionally include the ordinal position of one or more medications within a regimen 311; such ordinal responses may be numeric (e.g., first, second, third) or relative (e.g., first or last). Exemplary verbalized responses may also include an alternative name of a medication name 307 or the role of a medication 310, 314 (e.g., a premedication or post medication), as in a medication regimen.

Exemplary verbalized responses 302 generally and 304, 305, 307, 309, 310, 311, 312, and 314 specifically may also include one or more items (separately or in combination) selected from the set of the chemical name or generic name of one or more medications (e.g., dexamethasone, ipilimumab, or rituximab), the tradename of one or more medications (e.g., “Opdivo®” or “Herceptin®”), a shortened, colloquial, or “nickname” of one or more medications (e.g., “dex” for dexamethasone, “epi” for epinephrine, “nivo” for nivolumab, or “water pill” for furosemide or other diuretic medications), the concentration of one or more medications (e.g., “100 milligrams per milliliter” or “100 mg per mL”), the dose of one or more medications (e.g., “620 milligrams” or “620 mg,” “5 milligrams per kilogram” or “5 mg/kg”), the volume of one or more medications (e.g., “5 milliliters” or “5 mL”), a shortened, colloquial, or “nickname” of one or more parameters (e.g., “migs” for milligrams, “mils” or “ccs” for milliliters), the route of administration of one or more medications (e.g., “subcutaneously,” “SC,” “intravenously,” “IV,” “by inhalation”), the duration administration of one or more medications (e.g., “10 seconds,” “30 minutes,” “1 hour”), the rate of administration of one or more medications (e.g., “1 mL/minute,” “1 milliliter per minute,” “5 mg/kg/hour,” “5 milligrams per kilogram per hour”) the frequency or timing of administration of one or more medications (e.g., “daily” “monthly in the morning” “every 8 hours as needed”), or any other parameter or instruction that exists as part of a medication order or prescription.

### D. Illustrative Use Cases—Interrogating & Triaging Symptoms

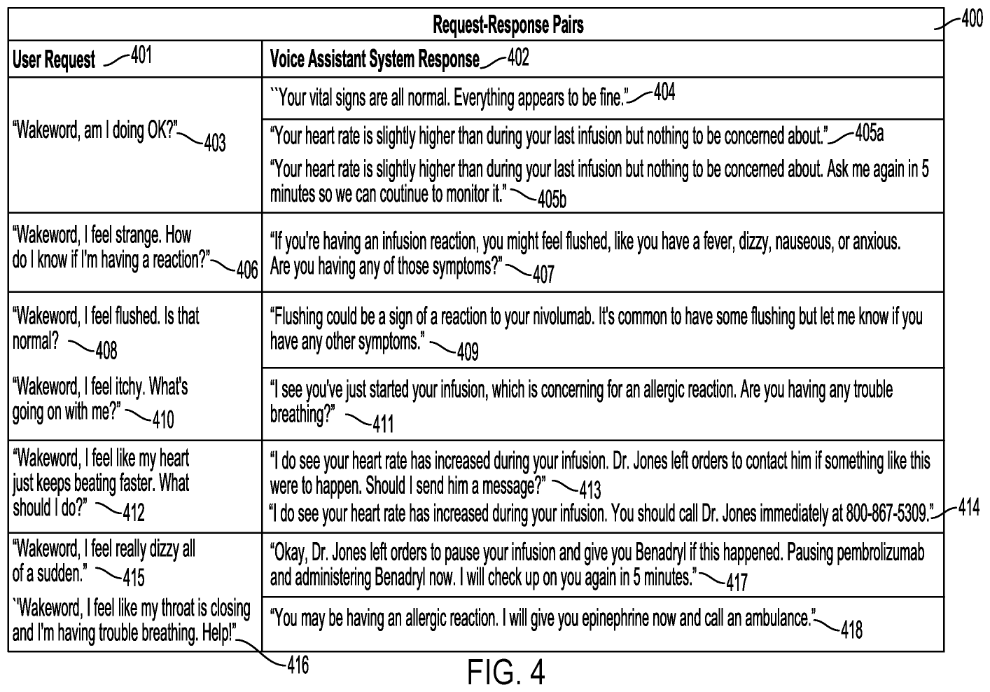

In some embodiments, the apparatus may be provisioned to allow interrogation of the voice assistant as to one or more unpleasant, uncertain, worrisome, or worsening physiologic symptoms experienced by a patient before, during, or after one or more medications delivered, being delivered, or to be delivered by the drug delivery system. FIG. 4 shows a series of exemplary illustrative pairs 400 of requests 401 and corresponding responses 402 related to the patient's current physiologic status in accordance with the embodiments described herein. In some embodiments, a user may interrogate the voice-enabled assistant as to a patient's current physiologic status as exemplified in requests 403, 406, 408, 410, 412, 415, and 416. Other aspects of physiologic status will be apparent to those skilled in the art based on the example requests 401, to which other responses may be formulated other than those exemplary responses 402 herein.

Such a request may include, for example, an inquiry as to real-time physiologic status 403 or a self-report of vague or indistinct symptoms 406, as might be present at the outset of a systemic infusion reaction. Such a request may also include an inquiry as how to interpret vague or indistinct symptoms 406. An exemplary verbalized response may comprise a response corresponding to a normal physiologic state 404. Some responses may include one or more reassurances to the user making the request, either based on current physiologic status 404, or comparison to a prior physiologic status or prior medication administration 405a. Some responses may optionally prompt a future revisiting of symptoms by the patient for continued monitoring 405b. A response may also comprise information to help distinguish adverse effects 407, optionally including one or more inquiries to a patient to ascertain the nature, severity, and/or urgency of the self-reported symptoms.

Requests may also include a self-report of specific symptoms 408, 410, 412, 415, 416, or a self-report of unexpectedly emergent, concerning, or worsening symptoms 412, 415, 416. Such a request may also include an inquiry as how to manage emergent symptoms 412, or request for assistance 416. An exemplary verbalized response 409 may comprise symptom triaging based on a report contained within the request 408, 410, for instance, providing reassurance that the symptoms reported are normal and to be expected. Alternatively, the response 411 may comprise contextual monitoring and direct questioning for differential diagnosis to ascertain the nature, severity, and/or urgency of the self-reported symptoms.

The context of a request, such as 412, 415, 416, may be indicative of one or more worsening physiologic parameters or symptoms of emergent or life-threatening concern. Based on the context of the request, an exemplary verbalized response may comprise one or more of acknowledging the self-report 413, contacting with a healthcare provider via the apparatus 413, instructing a user to contact a healthcare provider 414, or summoning emergency personnel 418. An exemplary verbalized response may also comprise an acknowledgement of progressive symptoms, optionally also communicating an escalation plan based on standing orders 413. Exemplary verbalized responses 417, 418 may also comprise a predefined action plan, emergency response protocol, or treatment guideline, as in the case of systemic infusion reaction or anaphylaxis, the responses being formulated based on one or more specific symptoms and/or symptom severity. Exemplary verbalized responses 413, 414, 417, 418 may also be formulated based on (and/or including) information contained in a patient's electronic health record system, for instance, a physician's standing order or medication order set referred to in responses 413, 417, or a standing order for epinephrine referred to in response 418. Exemplary verbalized responses 413, 414, 417, 418 may also be formulated based on (and/or including) information the context of the self-report, the stage of medication administration, the medication being administered, a prior history of symptoms, data from one or more physiologic sensors, or patient self-report. If the context of the symptoms or severity of symptoms are unclear from a patient's self-report, a response may also include one or more clarifying requests to a user to perform additional triage steps to provide further context and/or enable triaging of symptoms as described herein.

In some embodiments, a request such as 412, 415, 416 reporting emergent symptoms may also induce the apparatus to stop or slow an offending medication and notify a user of the apparatus 417, to administer a medication to counteract self-reported symptoms and notify a user of the apparatus 417, 418, or to summon emergency services and notify a user of the apparatus 418.

### E. Illustrative Use Cases—Drug Delivery Device Configuration

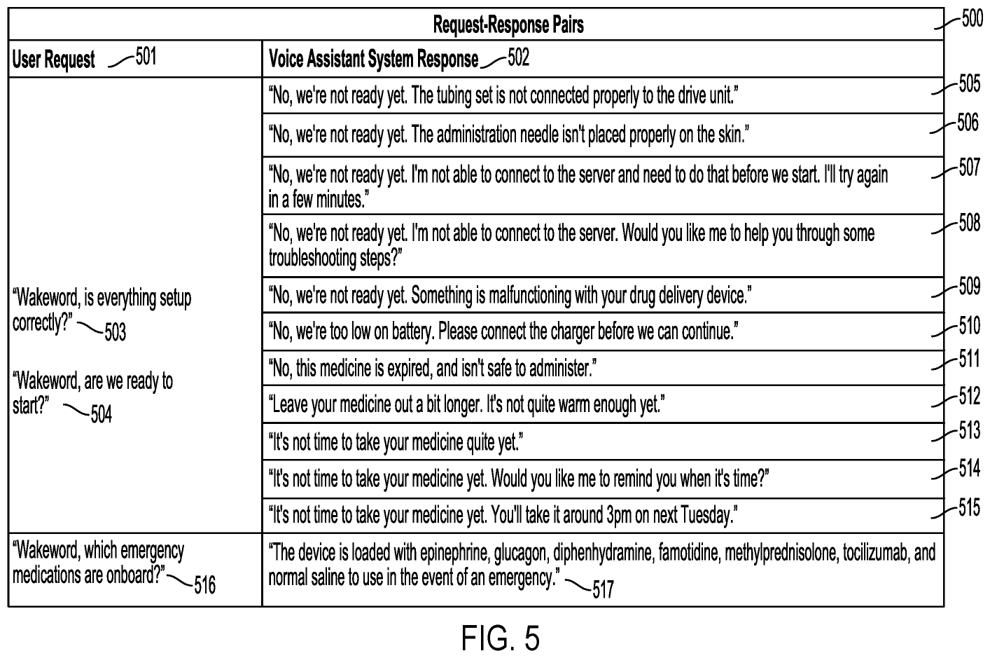

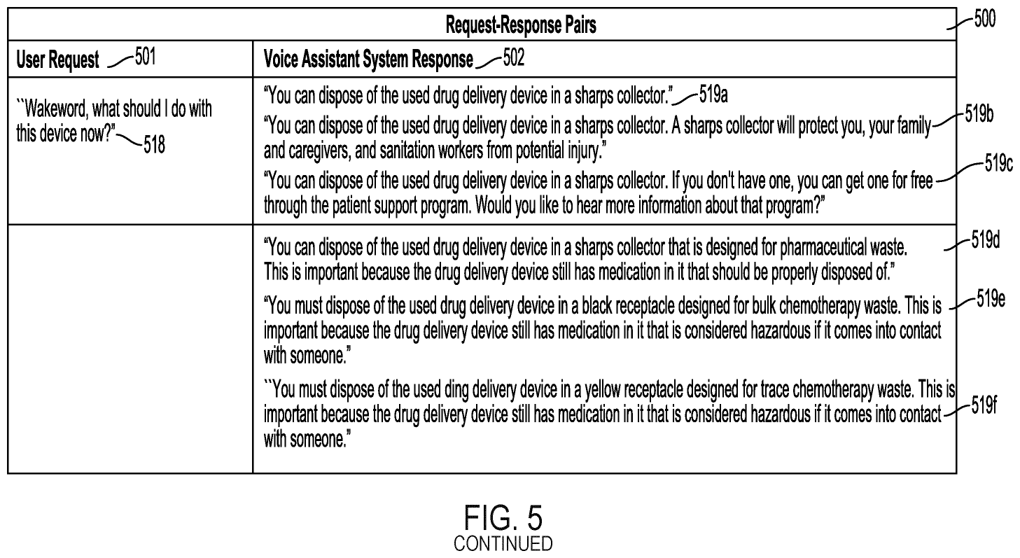

In some embodiments, the apparatus may be provisioned to allow interrogation of the voice assistant as to the readiness of the drug delivery device to be used by a patient or deliver one or more medications. FIG. 5 shows a series of exemplary illustrative pairs 500 of requests 501 and corresponding responses 502 related to the drug delivery device readiness for use in accordance with the embodiments described herein. In some embodiments, a user may interrogate the voice-enabled assistant as to the status of a drug delivery device as exemplified in requests 503, 504, and 515. Other aspects of drug delivery system status may be interrogated, as will be apparent to those skilled in the art based on the example requests 501, to which other responses may be formulated other than those exemplary responses 502 herein.

Such a request may include, for example, an inquiry to confirm proper setup of the delivery system components 503 and/or readiness to begin administration 504. In some embodiments, a request 501, 503 prompts a self-check of the apparatus to identify any erroneous configurations or aspects; in other embodiments, a self-check is performed when commanded by a user or by the controller of the apparatus. If drug delivery system components are incorrectly assembled or improperly placed, an exemplary verbalized response may comprise a recitation of the issue(s) with the specific components 505 and 506. If the drug delivery system components have lost or cannot establish connectivity, an exemplary verbalized response may comprise an error message 507 or an error message and troubleshooting workflow 508 provided by the drug delivery skill. If the drug delivery system components are damaged, malfunctioning, an exemplary verbalized response may comprise an error message 509. If the drug delivery system components do not have sufficient power, charge, or battery, an exemplary verbalized response may comprise a notification of power status and suggestion to charge the components 510. If the drug delivery system detects expired medication, an exemplary verbalized response may comprise a notification 511, optionally explaining the nature of the expiry and rationale for not using a medication in the system; further, such a state may induce the apparatus to prohibit medication delivery.

In one or more alternative embodiments, voice assistant responses 502 include one or more information or instruction related to components, use steps, conditions, error states, non-error states, and/or any aspects of a delivery system that a user may interact with during use, that may be misconfigured, or that may fail before, during, or after administration. Obviously, the context of request 502 shall dictate the responses 503, which may be adapted based on the type of drug delivery device being used with the apparatus, the medications contained within the apparatus, the configuration and/or setup steps required to use the device, or other relevant considerations.

Some medications may require refrigerated storage and warmup to room temperature prior to use; this may be advantageous for patient comfort, or to reduce the viscosity of a medication that becomes increasingly viscous at lower temperatures, such as a biologic medication. If one or more medications within the drug delivery system should warm to room temperature, but not have yet done so, an exemplary verbalized response to requests 503, 504 may comprise a notification 512, optionally including either a reminder or notification when the medication reaches the proper temperature, an explanation of how un-warmed medication may be uncomfortable, or an option to proceed with medication delivery despite not being fully warmed. If such confirmation is provided, the apparatus may optionally be configured to allow delivery of one or more un-warmed medications as described elsewhere herein.

Requests 503, 504 may be made inadvertently, for instance, by a user who does not understand or has forgotten the medication administration schedule. In such a case, the system may correct a user who has a misunderstanding of the medication schedule. An exemplary verbalized response for such a request may comprise a notification 513, a notification with offer of reminder 514, or a notification and correction as to the correct day and time 515. Alternatively, responses 513, 514, and 515 may also include an option for a user to administer one or more medications early, as may be required due to personal circumstances or healthcare provider instruction. Dependent on context and configuration of the apparatus, responses 513, 514, and 515 may also prompt a user for confirmation that a healthcare provider is aware of, and has approved, medication delivery to take place earlier than expected. If such confirmation is provided, the apparatus may optionally be configured to allow delivery of one or more medications as described elsewhere herein.

A user may also interrogate the voice-enabled system to confirm which emergency use medications are available for administration by the device 516. An exemplary verbalized response for such a request may comprise a recitation of one or more medications available for emergency use 517, optionally including one or more of dosing, concentration, route of administration, and administration conditions as described elsewhere herein.

In some embodiments, a user may interrogate the voice-enabled assistant as to how to safely dispose of a drug delivery device as exemplified in request 518. An exemplary verbalized response for such a request may comprise a recitation of safe disposal (as in a sharps collector if the drug delivery device has a needle) 519a, and may optionally explain the rationale for safe disposal to a user 519b. Alternatively, an exemplary verbalized response for such a request may include a prompt to obtain proper disposal implements (such as a sharps container) from a patient support program 519c, as are commonly provided by pharmaceutical companies. Obviously, the context of request 518 shall dictate the responses 519a, 519b, 519c, which may be adapted based on the type of drug delivery device being used with the apparatus, the medications contained within the apparatus, whether all medications have been administered, or whether unadministered medications remain in the device. As many countries have legislation on disposal of pharmaceutical waste, it may be advantageous to present an exemplary verbalized response such as 519d that instructs a user to dispose properly of a used drug delivery device that has unadministered medications, such as emergency medications described herein, or a drug delivery device that contains bulk 519e or trace 519f chemotherapy waste.

### F. Illustrative Use Cases—Emergency Response

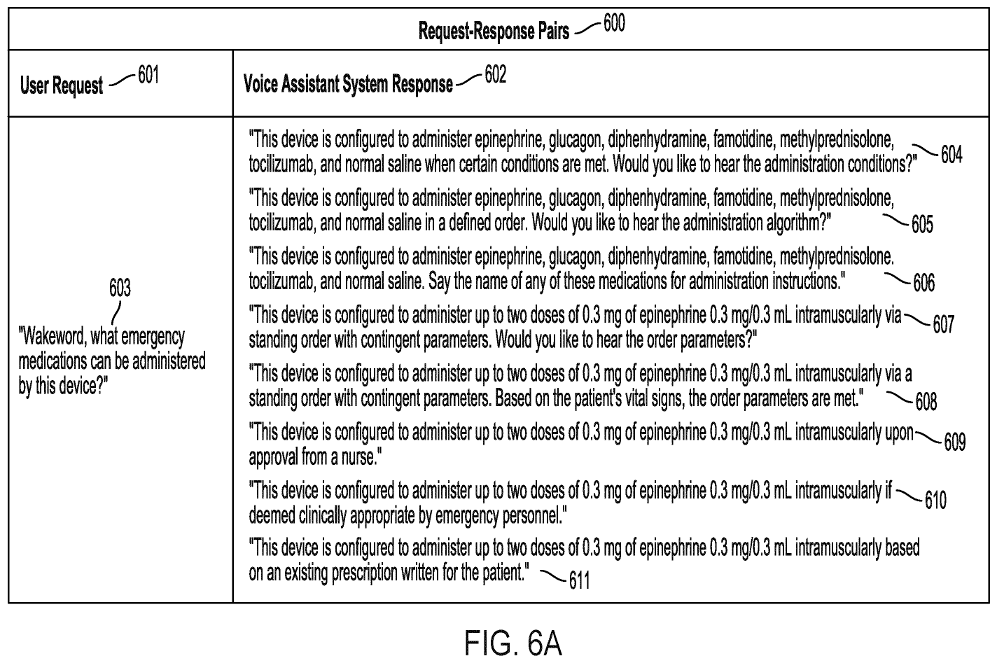

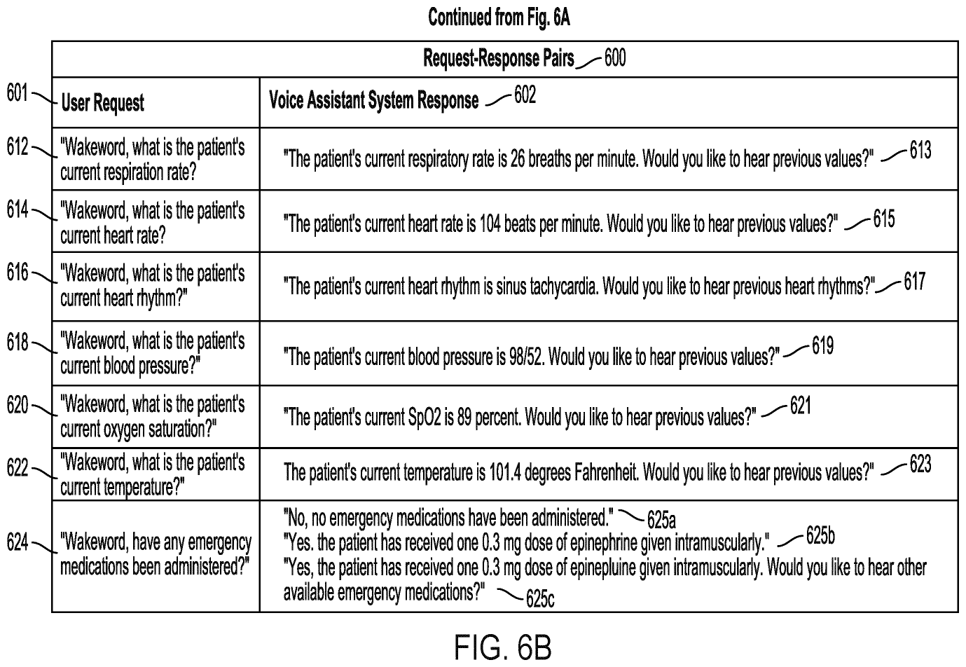

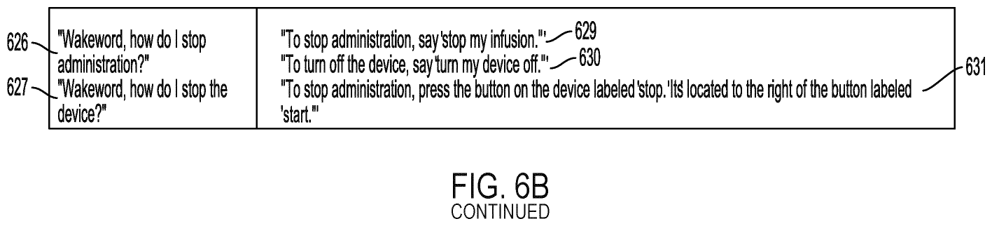

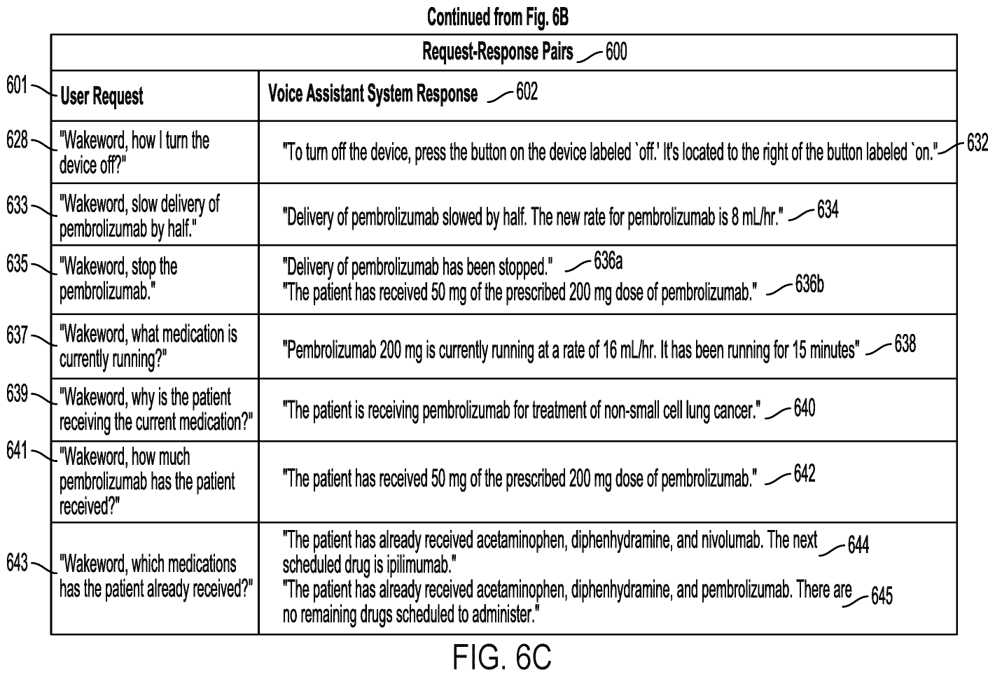

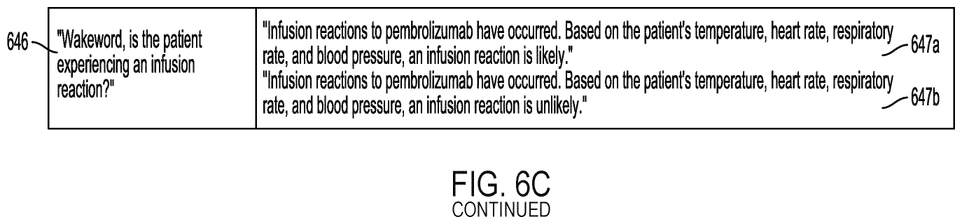

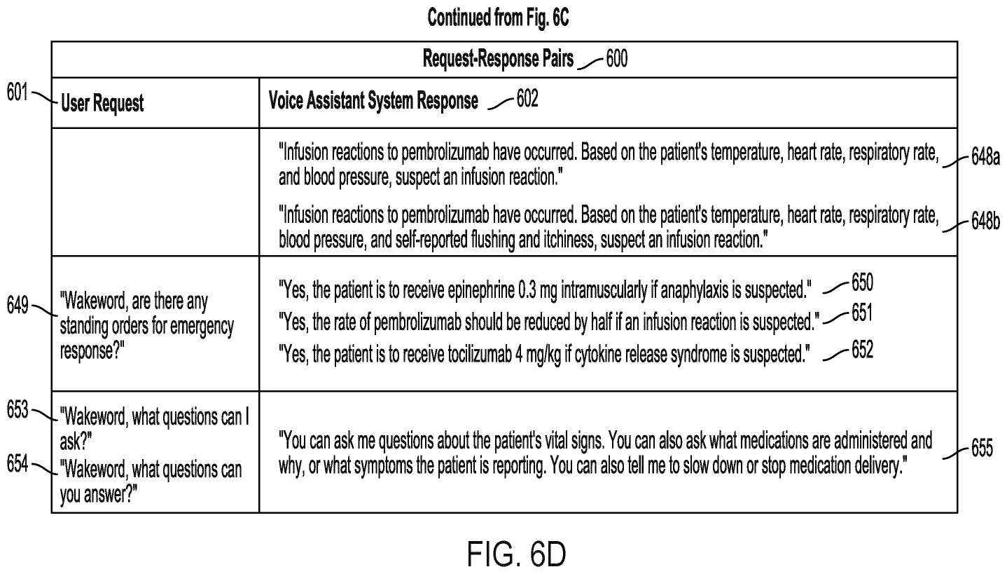

In some embodiments, the apparatus may be provisioned to allow interrogation of the voice assistant in an emergent situation, as in the case of a patient experiencing adverse reaction to a medication administered by the drug delivery system. FIGS. 6A-6D shows a series of exemplary illustrative pairs 600 of requests 601 and corresponding responses 602 related to emergency response in accordance with the embodiments described herein.

Requests 601 could be made by the patient themselves, a non-clinical caregiver providing care for a patient, or any healthcare worker providing care for a patient using the device, such as a nurse, pharmacist, doctor, hospice worker, social worker, home aide, ambulance worker, first responder, EMS technician, and the like. Additionally requests 601 are not limited solely to emergency personnel and may be made by anyone who may be called upon to render emergency care to a user of the drug delivery system, including an individual unfamiliar with the drug delivery system or apparatus.

In some embodiments, a user may interrogate the voice-enabled assistant as to medications configured within the device as exemplified in requests 603. Verbalized responses to request 603 may comprise a recitation of one or more of the current medications 604, 605, 606, 607, 608, 609, 610, 611, doses 607, 608, 609, 610, 611, administration route 608, 609, 610, 611, and administration instructions or conditions 608, 609, 610, 611. Exemplary verbalized responses to request 603 may comprise a prompt to a user to review one or more of the medication conditions 604, the administration algorithm or criteria 605, the administration instructions 606, the medication order parameters 607. Exemplary verbalized response 608 to request 603 may comprise a notification that one or more medications may be administered based on one or more medication order parameters. It will be apparent that many appropriate responses may be based on the medication, medication regimen, therapeutic area, prescriber preference, or patient situation.

In some embodiments, a user may interrogate the voice-enabled assistant as to medications configured within the device as exemplified in requests 612, 614, 616, 618, 620, 622. Exemplary verbalized responses to requests 612, 614, 616, 618, 620, 622 may comprise one or more physiologic parameters, including respiration rate 613, heart rate 615, an identification of heart rhythm 617, blood pressure 619, oxygen saturation 621, or temperature 623. It will be apparent that other physiologic, biologic, or biochemical parameters may be communicated in a similar fashion.

In some embodiments, a user may interrogate the voice-enabled assistant as to emergency medications administered by the device to a patient as exemplified in requests 624. Exemplary verbalized responses may include a confirmation that no medications have been administered 625a, or that an emergency medication has been given 625b, 625c. Based on the configuration of the medication delivery system, exemplary verbalized responses may also comprise a prompt to a user to review one or more emergency medications that are available for administration but have not yet been administered 625c.

In some embodiments, a user may interrogate the voice-enabled assistant to ascertain how to halt medication administration as exemplified in requests 626, 627, 628. Exemplary verbalized responses may comprise a reiteration of the voice command to halt administration 629 or turn off the drug delivery device 630, if such functionality is enabled as described elsewhere herein. Exemplary verbalized responses 631, 632 may also comprise instructions to halt administration by physically manipulating an aspect of the drug delivery device, as by a button or switch. Exemplary verbalized responses 631, 632 may optionally include guidance to a user as to the location of one or more physical aspects of the drug delivery device to speed an emergency response.

In some embodiments, a user may change the rate of drug delivery in an emergency, as exemplified in requests 633, 635. Exemplary verbalized responses may comprise a confirmation that drug delivery has been slowed 634 or stopped 636a. Exemplary verbalized responses 636b may optionally include an estimation of the partial dose of a medication delivered to a patient prior to the voice request 635.

In some embodiments, a user may interrogate the voice-enabled assistant to ascertain current parameters associated with medication administration, as exemplified in requests 637, 638, 641. Exemplary verbalized responses may include a recitation of the current medication, rate, and run time 638, of the indication for which treatment is being delivered 640, or the current dose delivered thus far 642. In some embodiments, a user may interrogate the voice-enabled assistant to ascertain current parameters associated with administration of a multi-medication regimen, as exemplified in request 643. Exemplary verbalized responses to request 643 may comprise a recitation of the medications administered to a patient and preview of the next drug to administer 644 or may comprise a recitation of the medications administered to a patient and confirmation there are no further drugs to administer 645.

In some embodiments, a user may interrogate the voice-enabled assistant to ascertain whether a patient receiving a medication is experiencing or suspected of experiencing a systemic infusion reaction as exemplified in request 646. Exemplary verbalized responses may, based on physiologic parameters, include confirmation that infusion reactions are likely 647a or suspected 648a, or may exclude infusion reaction 647b. Exemplary verbalized responses may optionally include patient-reported symptoms 648b, the patient-reported symptoms (and their ascertainment) being described elsewhere herein.

In some embodiments, a user may interrogate the voice-enabled assistant to ascertain what, if any, medication orders (i.e., a standing or contingent order by a physician) pertain to a patient in an emergency situation as exemplified in request 649. Exemplary verbalized responses to request 649 may comprise a recitation of one or more medications to be contingently administered according to a medication order 650, 652 or may comprise a recitation of one or more contingent changes to medication administration parameters according to a medication order 651. Exemplary verbalized responses 650, 651, 652 may optionally include a summary of the contingent circumstances under which medication administration is authorized by a medication order.

The foregoing requests may neither be apparent nor intuitive to an unfamiliar user of the apparatus. Thus, in some embodiments, a user may interrogate the voice-enabled assistant to ascertain what requests may be made to the system 653, 654. Exemplary verbalized responses 655 may include a summary of the requests that the apparatus is configured to respond to in an emergent situation. In one or more embodiments, the apparatus includes supportive materials so an unfamiliar user can activate and interrogate the apparatus in an emergency. For instance, supportive materials containing the wakeword 101, voice assistant activation 103 instructions, or one or more emergency request/response pairs (e.g., one or more exemplary request/response pairs 600/601, such as exemplary request/response pairs 652/655 and 653/655) may be placed in a central location familiar to emergency responders, such as a patient's refrigerator door. One or more supportive materials as described previously may be also provided on the display 110 of a voice assistant, optionally presenting the supportive materials in the form of step-by-step instructions to allow an unfamiliar device to activate, interrogate, or command the device. Supportive materials provided on display 110 may also be presented in a plurality of languages to enable a wide variety of users to render emergency care. In a preferred embodiment, verbalized responses 602 to requests 601 may vary in tone and content based on the context and nature of the request, or on the user anticipated to make a request of the system. Requests of a clinical nature receive a response spoken with a clinical tone, connotation, or content as exemplified by request/response pairs 620/621, 639/640, 646/647a and 647b, and 646/648a and 648b. Conversely, requests that may be made by a non-clinical user of the device receive a plainspoken, non-clinical tone and content, as exemplified by request/response pairs 633/634, 653/655 and 654/655.

In some embodiments, the system is configured to repeat one or more responses 602 multiple times by the voice assistant until acknowledged by a user of the apparatus through a verbalized response. In one or more embodiments, failure of a user to provide a response to the apparatus escalates the emergency response, contacting and/or notifying a healthcare provider, emergency personnel, or a designated emergency contact, such as a caregiver.

In some embodiments, the system is configured to deliver at least one spoken emergency response 602 in a plurality of languages. One of the multiple languages may be a default language configured within the voice assistant, the drug delivery skill, or the drug delivery device. Alternative languages may be predetermined based on the regions where the device will be used or may be selected to maximize comprehension in an emergent situation with an unfamiliar user of the device. For instance, a voice assistant using English as the default language may be configured to provide an emergency response 602 in English and Spanish. For instance, a voice assistant using German as the default language may be configured to provide an emergency response 602 in German, French, and Italian.

### G. Illustrative Use Cases—Patient Assessment

In some embodiments, the apparatus may be provisioned to allow the voice assistant to administer one or more patient reported assessments. Patient reported assessments or “assessments,” both terms of convenience used herein, refer to one or more questionnaires or survey instruments administered to a patient before, during, or after use of the apparatus herein. Patient reported assessments may be administered with the apparatus herein during clinical assessment, ongoing clinical care, as part of a clinical trial, as part of post-market surveillance activities mandated by pharmaceutical regulators, or as health outcomes economics research (“HEOR”) studies. They may also include, by way of example and not limitation, patient-reported outcomes (“PROs”), electronic patient-reported outcomes (“ePROs”), measures of disease symptoms and/or progression, measures of health behaviors, clinical trial questionnaires or diaries, or clinical assessments (whether validated or unvalidated). Patient reported assessments administered by the apparatus may cover a wide variety of topics; by way of example, assessments may be related to disease state status, health-related quality of life, treatment burden, mental health, or medication adherence. Patient reported assessments administered by the apparatus may also include more informal assessments used to inform a healthcare provider or caregiver as to the overall wellbeing, symptoms, concerns, or satisfaction of a patient using the apparatus. Patient reported assessments administered by the apparatus may also include non-clinical assessments, such as market research studies or tracking studies to understand product use (e.g., experience, practices, perceptions, satisfaction). Patient reported assessments administered by the apparatus may be conducted at a single point in time or may be repeatedly administered as a longitudinal assessment, where data from each interval may be compared and trended.

When giving assessments as described herein, the system first prompts the user with a question in each assessment, and the user of the system responds to each prompt with a response. The user's response may be to vocalize a numeric rating (e.g., as in a Likert scale), a descriptor (e.g., “Frequently,” “Sometimes,” “Almost Never,” or “Infrequently”), or closed-form response from a range of options.

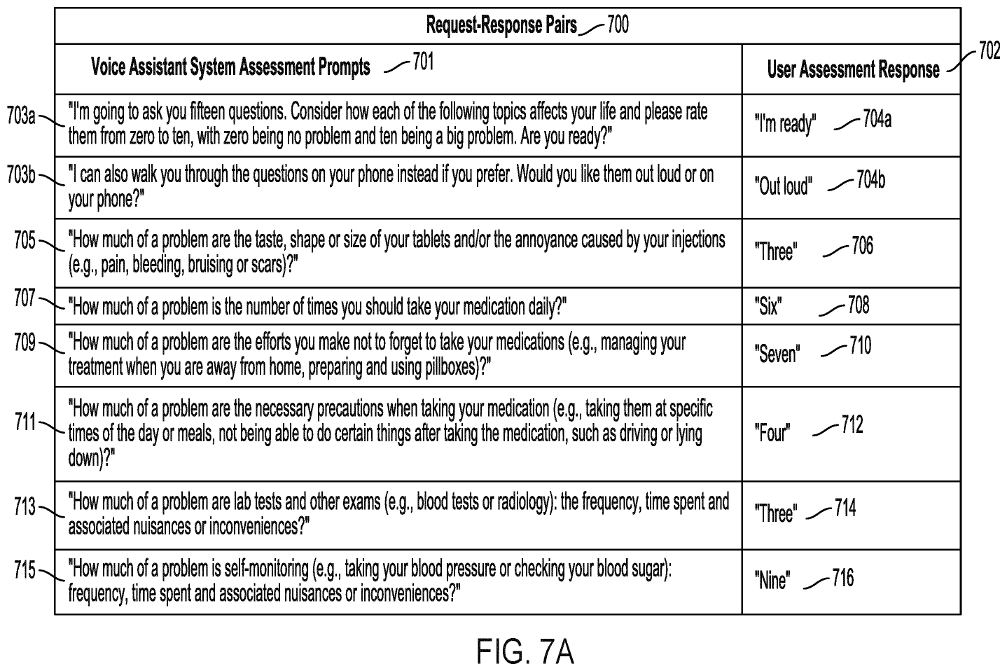

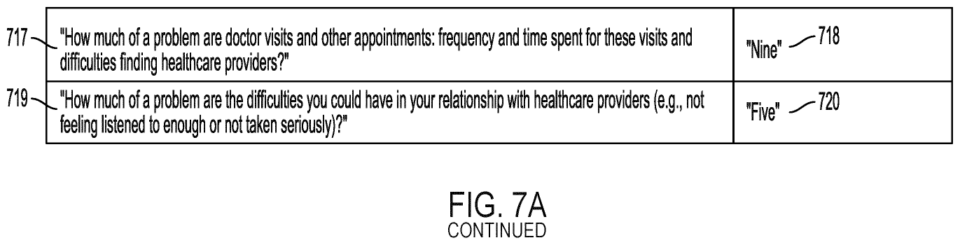

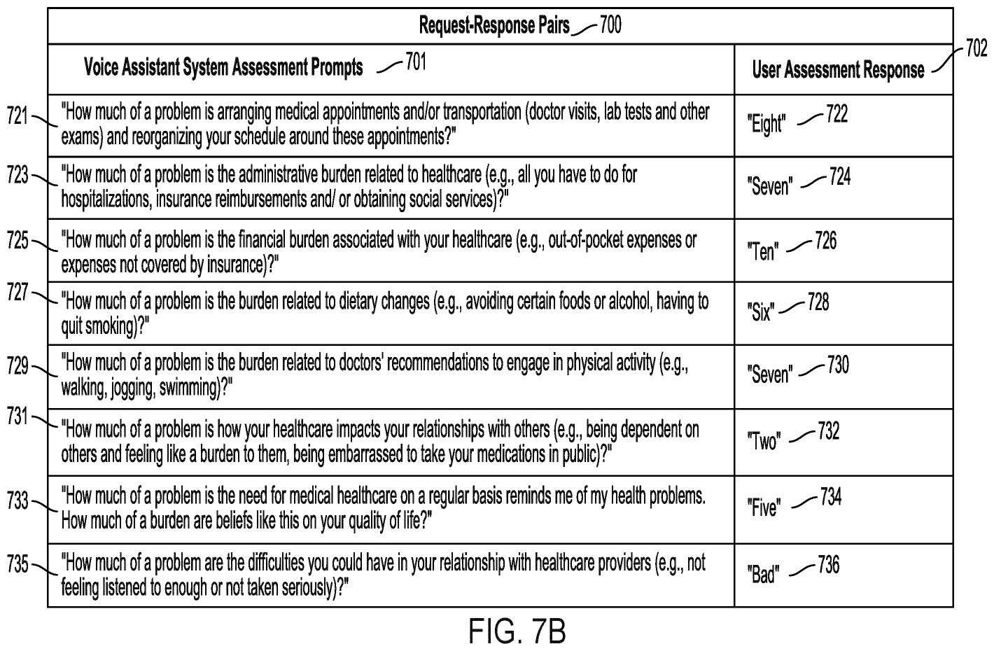

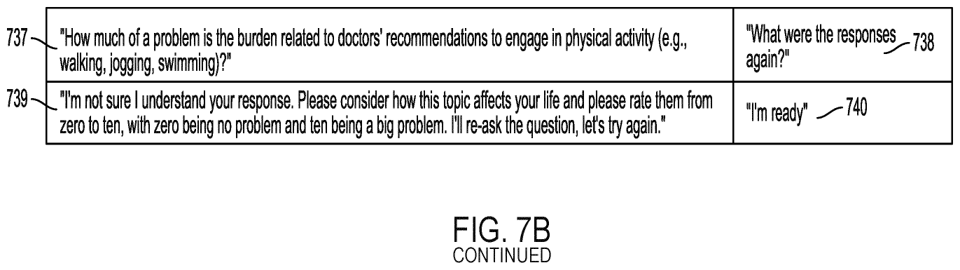

FIGS. 7A-7B shows a series of exemplary pairs 700 of assessment prompts 701 and corresponding responses 702 illustrating administration of the Treatment Burden Questionnaire (TBQ), a validated clinical assessment of chronic treatment burden faced by a specific patient. The TBQ features fourteen questions asked in succession to a patient, each question corresponding to specific domains of treatment burden, and each question receiving a rating from a patient on a ten-point Likert scale. The TBQ is provided as an exemplary embodiment and shall not be construed as limiting the apparatus to a specific questionnaire or response scale(s).

The system presents an introductory prompt to a user 703 giving background regarding the assessment and response scale to be used. The user may indicate they are ready to begin 704a; alternatively, they may defer the assessment to a later time. Exemplary pairs assessment prompt/response pairs 703a/704a, 703b/704b, 705/706, 707/708, 709/710, 711/712, 713/714, 715/716, 717/718, 719/720, 721/722, 723/724, 725/726, 727/728, 729/730, 731/732, 733/734, and 735/736 are sequentially asked by the system and answered by a user.

In some embodiments, the system is configured to respond to a user who gives impermissible responses or may be confused during an assessment being administered by the system. For instance, an assessment prompt 735 may be administered, to which the user may provide an impermissible response 736 (i.e., responding “Bad” when the appropriate response comprises a numeric rating from 1-10); the system may respond with a reiteration of the rating scale and/or introduction (e.g., assessment prompt 703) or other corrective instructions. Similarly, in some embodiments, the system is configured to respond to a user who may be confused during an assessment being administered by the system. For instance, an assessment prompt 737 may be administered, to which the user may indicate in their response confusion about the permissible responses 738; the system may respond with a reiteration of the rating scale and/or introduction (e.g., assessment prompt 703) or other corrective instructions 739, to which the user may optionally provide a response indicating they are ready to continue 740. In some embodiments, the system may also be configured to accept a close approximation of the options presented. For instance, if one of the ratings were “Worst pain I've ever experienced,” the system may accept verbatim responses (e.g., “Worst pain I've ever experienced”) or an abbreviated response (e.g., “Worst pain,”). The system may provide instructions at the outset of an assessment or remind a user about the permissible responses.

Once all assessment prompts are successfully completed, verbalized assessment responses 704a, 704b, 706, 708, 710, 712, 714, 716, 718, 720, 722, 724, 726, 728, 730, 732, 734, 736 are processed by the voice assistant and drug delivery skill as are other verbalized responses as described herein, generally comprising conversion of a spoken response to data corresponding to the spoken response. For instance, the TBQ data would be converted into numeric data corresponding to the spoken response to each assessment prompt, then scored by adding a respondent's scores, yielding a TBQ score from 0-140, higher scores indicating higher treatment burden. Although the TBQ consists of questions on a ten-point Likert scale, nothing in the disclosure shall be construed as limiting the scope of the disclosure herein to a specific response type or rating scale; to the contrary, the apparatus herein, owing to the flexibility of the voice assistant, voice service, and drug delivery skill, is able to accommodate many different patient reported assessments and many different rating scales.

In one or more embodiments, scoring of a patient reported assessment may be performed by the drug delivery skill, drug delivery device, or an external computing device and/or service. In some embodiments, data from the assessment may be stored in memory on the voice assistant, drug delivery skill, drug delivery device, externally connected database, electronic health record system, clinical trial data storage system, or other database.

The system may be configured to administer assessments at various points before, during, and after medication administration using the apparatus. In some embodiments, the system may prompt a user to complete an assessment during medication administration with the apparatus, thus taking advantage of the time required for administration. In some embodiments, the system is configured to administer an assessment with a plurality of portions, each portion corresponding to a different aspect of medication administration. In some embodiments, the system is configured to administer a two-part assessment to a user of the apparatus, the first part being administered before medication administration begins, and a second part being administered after medication administration completes. For instance, a two-part assessment is the SIAQ (Self Injection Anxiety Questionnaire), comprising a pre-injection portion and a post-injection portion, the two portions being collectively and numerically scored after a given medication administration event, such as an injection or infusion. Other such assessments will be apparent to those skilled in the art.

The system may be configured to administer assessments according to a predefined schedule, on an ad hoc basis in response to a user request, based on sensor data received by the apparatus, or when specific medications are administered by the apparatus.

In some embodiments, assessments are administered according to a schedule corresponding to one or more of a predefined medication administration interval, a predefined schedule predefined by a healthcare provider, or a predefined schedule contained in a clinical study trial protocol or market research study. In some embodiments, a user of the apparatus may initiate an assessment based on a verbalized request to a system at a time of the user's own choosing.

In some embodiments, assessments are administered when a specific medication and/or medication regimen is being administered (or has been administered) by the device. In some embodiments, assessments collect information directly related to the drug(s) delivered by the drug delivery device, thus capturing data on the patient's status before, during, or after drug administration.

In some embodiments, the apparatus is configured to administer an assessment in response to sensor data collected by the drug delivery device and/or apparatus, the sensor data indicating medication administration is set to begin or is in process. In some embodiments, the apparatus administers an assessment in response to sensor data passively collected by the drug delivery device or associated sensors, the sensor data indicating a potentially abnormal device state, drug delivery status (e.g., ongoing, completed, paused), a change in patient status, symptoms, or activity, or a change in medication-taking behavior.

### H. Language & Speech Accommodation

While the foregoing examples of requests and responses are provided in English for purpose of illustration, nothing herein shall be construed as confining the apparatus to use of the English language, as the apparatus described herein may be configured to flexibly interact with users in their preferred and various languages. In one or more embodiments, the apparatus is configured to receive a request (e.g., 301, 401, 501, 601, 702) and formulate a response (e.g., 302, 402, 502, 602, 701) in one or more languages supported by the apparatus. In one or more embodiments, the apparatus comprises a voice assistant system capable of receiving requests and/or providing responses in one or more different languages and/or linguistic dialects of the same language. In one or more embodiments, the configuration of the voice assistant system used with the apparatus determines the permissible language(s) used by the apparatus for receiving requests and issuing responses. In one or more embodiments, permissible language(s) used by the apparatus for receiving requests and issuing responses are configured during manufacture of one or more of the drug delivery device, the drug delivery skill, or the voice assistant system. In one or more embodiments, permissible language(s) used by the apparatus for receiving requests and issuing responses are configured during dispensing of the apparatus to a patient. In one or more embodiments, permissible language(s) used by the apparatus for receiving requests and issuing responses are configured based on language preferences for a patient, the language preferences being contained in an electronic health record system for said patient. In one or more embodiments, permissible language(s) used by the apparatus for receiving requests and issuing responses are configured through use of a companion application, such as a mobile phone application. In one or more embodiments, permissible language(s) used by the apparatus for receiving requests and issuing responses are configured during installation and initial setup of the drug delivery skill. In one or more embodiments, permissible language(s) used by the apparatus for receiving requests and issuing responses are re-configured (i.e., changed) during installation and initial setup of the apparatus.

In one or more embodiments, at least of one of the languages used for the response is the language used to issue the request (e.g., both request and response in German). In one or more embodiments, one or more of the languages used for the response is different than the language used for the request (e.g. request in German, response in German and English). In one or more embodiments, a request-response pair comprises a request in a first language (e.g., English) and a response in one or more languages (e.g., English and Spanish; or English, Spanish, German, and French; or Spanish, German, and French).

In one or more embodiments, the apparatus is configured to identify the language used in a request and adaptively formulate a spoken response in the same language used for the request (e.g., a first request made in English receives a response in English, while a second request in Japanese receives a response in Japanese). In one or more embodiments, the apparatus associates a primary language for each user of the apparatus, the primary language subsequently used by the apparatus to issue responses. In one or more embodiments, the apparatus establishes the primary language based on the language of a user's first request to the apparatus. In one or more embodiments, the apparatus establishes the primary language during the configuration of a user's authentication voiceprint or passphrase as described elsewhere herein.

### I. Illustrative Use Cases—Authenticating Control of the Drug Delivery Device

The apparatus and methods described herein may also be used to control the delivery device, either as a primary means of control, or supplementary to other methods of control, such as activation features on the device itself or companion mobile phone applications. When voice interactions are used to control a drug delivery device, numerous medication safety considerations become paramount, as drug delivery devices may be interrupted inadvertently or directed to act in ways that may create a hazardous situation or cause patient harm. In addition, use of a voice assistant may raise patient privacy considerations, as patient-specific information may be verbally articulated by the apparatus during use that is subject to protection by government privacy regulations (e.g., the United States Health Insurance Portability and Accountability Act of 1996 [HIPAA], or the European Union General Data Protection Regulation [GDPR]). In addition, compliance with these privacy regulations (or other regulations) may be required to connect or interface a drug delivery apparatus, such as that described herein, with other healthcare systems, such as electronic health record (EHR) systems, patient monitoring equipment, or patient wearable databases. Even in the absence of a specific binding government privacy regulation, patients may desire to protect privacy of their health-related information and restrict personal information that is shared with others. Thus, in another alternative embodiment, the voice-enabled assistant described herein is provisioned to only respond to inquiries from one or more authorized persons to ensure one or more of medication safety, patient safety, or patient data privacy.

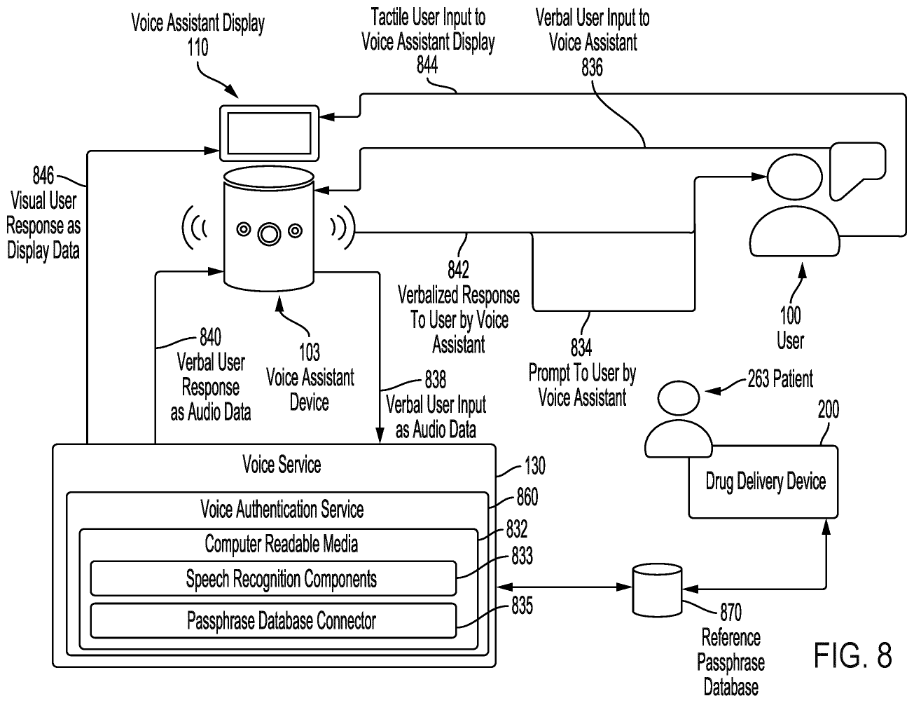

In an alternative embodiment, the apparatus is configured to recognize a specific authorized user the apparatus herein based on a correctly spoken passphrase. Referring to FIG. 8, in one or more alternative embodiments of the apparatus comprises a voice assistant device 103 with optional voice assistant display 110 and voice service 130 as previously described, with voice authentication service 860 configured to establish and store an authentication phrase for one or more authorized users of a device. Voice authentication service 860 comprises computer readable media 832, speech recognition components 833, a passphrase database connector 835, and optionally, a reference passphrase database 870. In one or more embodiments, speech recognition component 833 is shared with speech recognition component 133 present in the voice service as described previously. In one or more embodiments, speech recognition component 833 is configured to process, analyze, or compare voiceprint data (e.g., audio waveform data) from a user of the apparatus.

In one or more embodiments, the authentication process is initiated by the voice assistant device 103 through issuance of a prompt 834 provided to a user of the device. In one or more embodiments, prompt 834 is provided to a user of the device as a part of an initial configuration process of the apparatus. In one or more embodiments, the authentication process is initiated by the voice assistant device 103 in response to a query or command issued to the apparatus. In one or more embodiments, the authentication process is initiated by the voice assistant device 103 in response to a request from a user 100 of the apparatus and/or a patient 263 using the drug delivery device 200.

Referring again to FIG. 8, in one or more embodiments, voice assistant device 103 verbally prompts 834 a user 100 to recite a passphrase; user's recitation 836 is recorded by the voice assistant device 103 and communicated as input audio data 838 to the voice service 130 and in turn, the voice authentication service 860. In some embodiments, prompt 834 may instruct a user to recite a phrase of their choice or to recite a predetermined phrase. The voice assistant device 103 may in turn provides a verbalized response 842 to the user 100.

In or more embodiments, the passphrase may be prompted audibly by the voice assistant device 103. In or more embodiments, the passphrase may be prompted visibly by the voice assistant device 103 on an optional voice assistant display 110. In such an arrangement, tactile user input 844 may be provided to the voice assistant display 110 by the user 100. The voice service 130 may provide visual user response display data 846 to the voice assistant display 110. In some embodiments, the prompt may optionally include contextual information about how the information supplied will be used or why a passphrase is required. In one or more embodiments, the passphrase may be provided on one or more of the apparatus, components of the apparatus, packaging for one or more components of the apparatus, or instructional materials for the apparatus.

Some embodiments of the apparatus are configured to recognize a specific authorized user of the device based on individualized biometric aspects of the authorized user's own voice, such as a voiceprint. In one or more embodiments, audio data 840 is analyzed by the voice authentication service 860 to identify individualized biometric aspects of the user's own voice, which may be stored in a reference passphrase database 870. Individualized biometric aspects may include one or more of as-recorded (“raw”) audio data, processed versions of the as-recorded audio data, or intonation, as non-limiting examples. In some embodiments, there may be an option to offer a second predetermined passphrase if a user cannot articulate the first one (e.g., due to an accent or pronunciation issues).

In one or more embodiments, the voice service 130 and speech recognition components 833 are configured to evaluate the quality of audio data 840 and thereby assess said data's suitability for use as a future reference.

Some embodiments of the apparatus are configured to recognize a specific authorized user of the device based on correct recitation of a passphrase. In one or more embodiments, audio data 840 is analyzed by the voice authentication service 860 or speech recognition components 833 within the voice authentication service 860 (as described previously) to identify the spoken words associated with the user's passphrase and store audio data as text, which may be stored in a reference passphrase database 870. The voice authentication service 860 may provide confirmation of a successful passphrase, or may offer an option to retry if the audio data 838 is unintelligible. In one or more embodiments, the apparatus is configured with one or more predetermined passphrases and a confirmatory process to ensure the user can successfully verbalize the passphrase prior to use of the apparatus.

In one or more embodiments, the spoken passphrase is stored by the apparatus in reference passphrase database 870 without confirmation by the user 100. In one or more embodiments, the spoken passphrase is stored by the apparatus in reference passphrase database 870 only after successful confirmation takes place with the user 100. In one or more embodiments, a confirmatory process is included before the proposed user passphrase is established by the apparatus. In one or more embodiments, the proposed user passphrase is passed to the drug delivery skill 134, the skill formulating a confirmatory response as data incorporating the proposed phrase (e.g., “Your passphrase is I like fast cars. Is that correct?”) to the voice service 130.

The voice authentication service 860 may store information levels associated with one or more levels of access control for the device. Such information levels may include one or more levels associated with queries that control the device and those that do not. Such information levels may further include those that incorporate emergency commands and those that do not incorporate emergency commands. Further, certain levels of access control may require a spoken passphrase, while other levels of access may require two-factor authorization (e.g., using both a spoken passphrase and a confirmation on a separate device such the user's phone or computer). In one or more embodiments, the apparatus is configured to store redundant copies of reference passphrase database 870. In one or more embodiments, redundant copies of reference passphrase database 870 may be stored on one or more of the voice assistant device 103, voice authentication service 860, drug delivery device 200, or a companion application (e.g., mobile phone app) used with the drug delivery device 200.

In one or more embodiments, the apparatus is configured to require all users to recite the same passphrase (e.g., “My voice is my password”). In one or more embodiments, the apparatus is configured to store one or more passphrases, each passphrase corresponding to one or more authorized users of the device.

In one or more embodiments, the passphrase consists of a single word that is easily memorable by a patient (e.g. “Marrakesh”). In one or more embodiments, the passphrase comprises a personalized phrase selected by each user of the apparatus. In one or more embodiments, the personalized passphrase is a full sentence or phrase (e.g., “I like fast cars”). In one or more embodiments, the personalized passphrase is a series of words (e.g., “browse-bismuth-persian”) that do not constitute a grammatically correct phrase or sentence. In one or more embodiments, the passphrase consists of a simple commonplace phrase, colloquialism, or aphorism that is easily memorable by a patient (e.g., “A dime a dozen” or “A piece of cake”). In one or more embodiments, the passphrase consists of a relevant personal phrase (e.g., “Erika is my wife”or “My wedding day was the happiest day of my life”).

In some embodiments, the passphrase is intentionally selected to make verbalization, memorization, or repetition by children difficult or impossible, adding an element of child-resistance to the system. In some embodiments, the child-resistant passphrase is constructed of words selected specifically to make verbalization or memorization difficult for younger children (e.g., “browse-bismuth-persian”). In some embodiments, the child-resistant passphrase is constructed of long, phrases (e.g., “the sly fox jumped over the lazy dog”) that are selected specifically to make verbalization or memorization difficult for younger children. In some embodiments, the child-resistant passphrase is constructed of words above a specific reading level for a specific language used by the passphrase. In some embodiments, the child-resistant passphrase is constructed of words that are that are provided at or above a specific reading level for a specific language of choice, said words also tested by a method for readability (e.g., the passphrase “nicely caring individual,” is scored as a 15^{th} grade US English reading level on the Flesch-Kincaid readability scale).

In one or more embodiments, the passphrase is selected by an individual authorized user (e.g., “I'm Larry, and Jane is my mother” or “I'm Sue, and Jane is my mother-in-law”). In an alternative embodiment, the passphrase corresponds to an authorized user's role (e.g., “I'm Chris, Emmi's nurse,” or “I'm Erika, Emmi's daughter [caregiver],” or “I'm Larry, and Jane is my mother,” or “I'm Sue, and Jane is my mother-in-law”). In an alternative embodiment, the passphrase corresponds to the authorized user's affiliation (e.g., “I'm Joe with Atlantic Health System.”).

The apparatus described herein may be subject to one or more governmental regulations related to medical device quality assurance, including diagnosis of device failures and evaluation of received product complaints from patients or healthcare providers. Thus in an alternative embodiment, one or more passphrases comprise a concealed diagnostic passphrase embedded in the apparatus at the time of manufacture (e.g., “I'm in quality assurance with DeviceCo”) and/or dispensing to a patient (e.g., “I'm in biomedical engineering at Atlantic Health System”). In one or more embodiments, a concealed diagnostic passphrase is used to access diagnostic features of the apparatus during manufacture, quality assurance activities, post-market surveillance, or complaint analysis. In one or more embodiments, a concealed diagnostic passphrase is used to access diagnostic features of the apparatus in conjunction with a second authentication method, such as two-factor authentication or use on a one-time password. In one or more embodiments, diagnostic features of a concealed diagnostic passphrase may allow a user access to data contained in the apparatus that is not normally exposed to user in ordinary query, command, or use of the apparatus herein.

In some embodiments, the drug delivery system is configured to detect fraudulent attempts to configure and/or access the system. In some embodiments, the drug delivery system is configured to detect fraudulent attempts to access the system via a fabricated, counterfeit, or mimicked voice inputs during either or both of configuration and/or access. In some embodiments, configuration to detect fraudulent attempts to access the system include detection of synthetic speech, pre-recorded voice, or compositions of pre-recorded voice clips. In some embodiments, configuration to detect fraudulent attempts to access the system include notifications to a clinician, caregiver, or other third part. In some embodiments, configuration to detect fraudulent attempts to access the system include logging said attempts by the drug delivery system. In some embodiments, configuration to detect fraudulent attempts to access the system include disabling one or more request-response pairs provided within the drug delivery system. In some embodiments, configuration to detect fraudulent attempts to access the system include disabling one or more request-response pairs that control one or more aspects of the drug delivery system. In some embodiments, configuration to detect fraudulent attempts to access the system include disabling one or more request-response pairs that allow query as to the state of one or more aspects of the drug delivery system. In some embodiments, configuration to detect fraudulent attempts to access the system include disabling one or more request-response pairs that allow query as to the state of one or more aspects of the patient receiving medication from the drug delivery system. In some embodiments, configuration to detect fraudulent attempts to access the system include disabling one or more request-response pairs while enabling one or more request-response pairs, such as request-response pairs related to emergency situations and/or care.

In some embodiments, the apparatus is configured to detect fraudulent attempts to configure and/or access the system by a domesticated or household animal/pet present in the user's home (e.g., a cat or parrot) to inadvertently activate or command the voice assistant through mimicry. In some embodiments, the passphrase is selected to avoid mimicry by a domesticated or household animal/pet present in the user's home (e.g., a cat or parrot). In one or more embodiments, speech recognition components 833 are configured to analyze recorded passphrase data and detect potential mimicry by signal analysis or probabilistic methods. In one or more embodiments, the configuration process for the apparatus includes one or more confirmatory questions related to the presence of an animal/pet that can mimic voice commands, the one or more confirmatory questions being used to selectively enable and/or disable one or more voice commands the apparatus may respond to.

While the foregoing examples of passphrases are provided in English for purpose of illustration, nothing herein shall be construed as confining passphrases to English, and it will be apparent to those skilled in the art that the present disclosure may be used for any spoken language. In one or more embodiments, all authorized users of the device use a selected passphrase in a single language (e.g., US English, Swiss German, German, or French). In one or more embodiments, each user of the device may record a passphrase in their preferred language. In one or more embodiments, the passphrase is selected to be easily recorded, analyzed, and approved despite accented speech. In one or more embodiments, the passphrase is selected to exclude words that may be misheard or misinterpreted with accented speech in a non-native language.

### J. Illustrative Use Cases—Interrogating the Drug Delivery Device & Drug Delivery Process

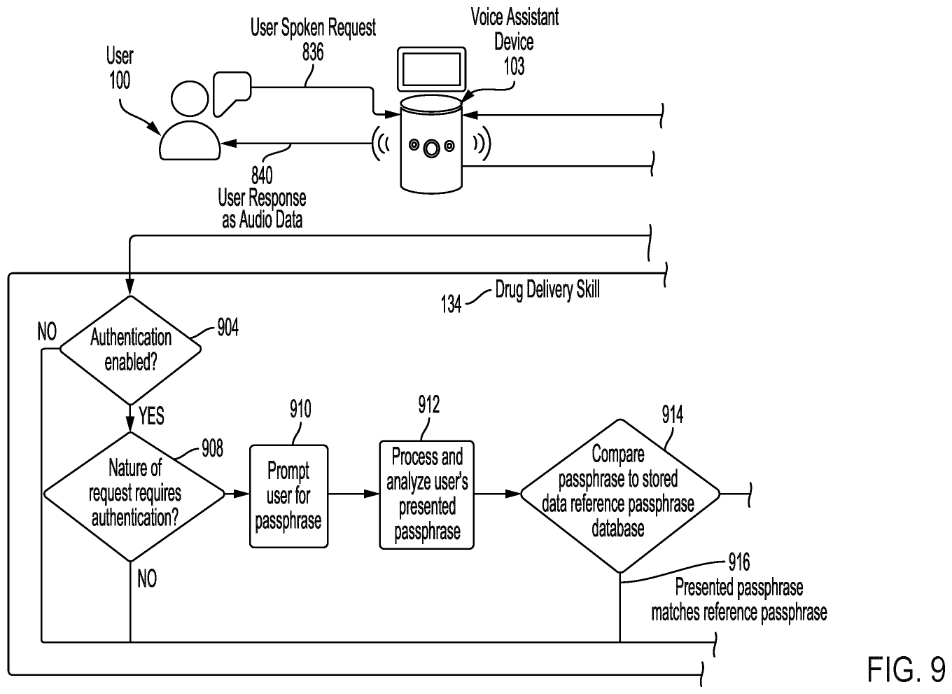

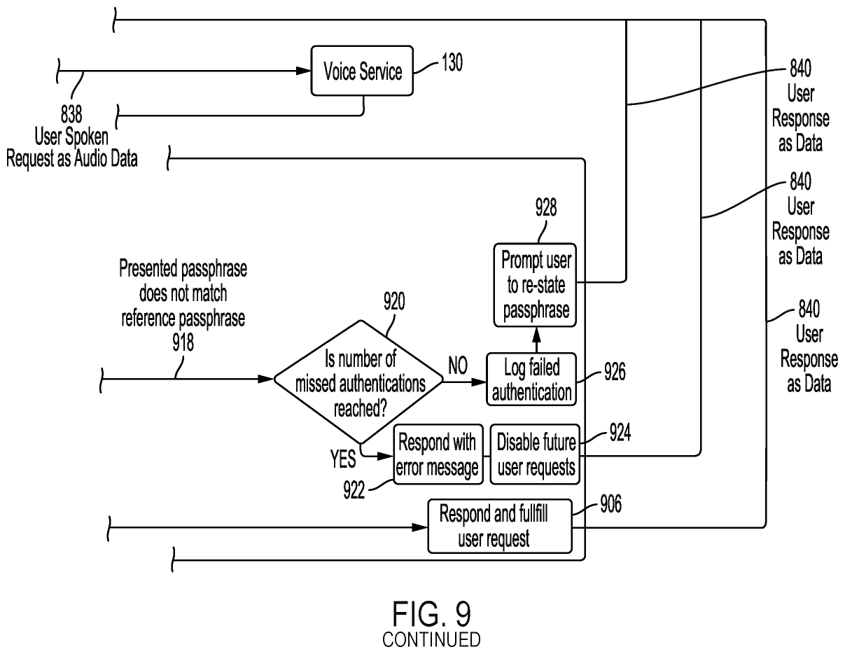

In some embodiments, the drug delivery system is configured to authenticate a user of the device prior to fulfilling a response made to the drug delivery system using the apparatus described herein. Referring now to FIG. 9, a user 100 makes a request 836 of the voice assistant related to the drug delivery system, which is relayed as audio data 838 to the voice assistant service 130 to parse intent and context of the request as described previously, which is then relayed to the drug delivery skill 134. At block 904, the drug delivery skill 134 determines whether authentication is enabled. If not, the drug delivery skill 134 responds and fulfills the user's request 906. If authentication is enabled, at block 908 the drug delivery skill 134 next evaluates whether request 836 requires one or more permissions enabled by the security controls described herein, namely a voiceprint or passphrase.

If the request 836 does not require advance authentication (e.g., an inquiry as to the status of the device, such as “How much time is left in my infusion?”), the drug delivery skill 134 interrogates the drug delivery device 902 to obtain the relevant parameters, which are in turn communicated 906 to the drug delivery skill 134. The drug delivery skill formulates data corresponding to a natural language response (e.g., “32 minutes are left in your infusion”) and returns said data 840 to the voice assistant device 103 which provides a verbalized response to the user 100.

If, however, the request does require advance authentication (e.g., a request to control the device, such as “I'd like to speed up my infusion”) the drug delivery skill 134 first formulates a response prompting the user to authenticate themselves by providing the predetermined passphrase. If a voiceprint is used, the passphrase is prompted 910, recorded when uttered by the user 100, and compared to the reference voiceprint. If a passphrase is used, the text is processed an analyzed at block 912, as discussed in additional detail above. At block 914, the drug delivery skill 134 compares the processed passphrase to a stored data reference passphrase database. If there is a match 916, the drug delivery skill fulfils the request as described elsewhere herein. If there is a mismatch 918, the user may be prompted one or more times to re-utter their passphrase, repeating the process, or the drug delivery skill may notify the user that they are unable to fulfill the request.

In one or more embodiments, at block 920 the drug delivery skill 134 evaluates whether a number of missed authentications exceeds a threshold. If the number of missed authentications exceeds the threshold, the drug delivery skill may respond with a error message 922 and/or disable future user requests 924 for a time period. Such failed authorization events are logged in memory or storage by one or more of the drug delivery device 200 drug delivery skill 134, or voice assistant device 103. In one or more embodiments, the drug delivery skill 134 makes one or more request-response pairs requiring authentication unavailable after a threshold value of failed authorization attempts is recorded by a user 100 of the apparatus. In one or more embodiments, the threshold value of failed authorization attempts comprises a total cumulative integer count. In one or more embodiments, the threshold value of failed authorization attempts comprises a total cumulative integer count per predefined time period (e.g., 1 day, 30 minutes, or 6 hours). In one or more embodiments, the threshold value of failed authorization attempts comprises a preset value configurable in one or more of the voice assistant device 103, drug delivery device 200 or drug delivery skill 134. In one or more embodiments, the threshold value of failed authorization attempts comprises a preset value configured by either of a patient using the drug delivery device 200 or a clinician prescribing, dispensing, or using the drug delivery device 200. In one or more embodiments, the drug delivery skill 134 ignores the request 836 if the user cannot be authenticated. In one or more embodiments, the drug delivery skill 134 comprising a notification to the user 100 comprising troubleshooting suggestions if the user 100 cannot be authenticated.

If the number of missed authentications does not exceed the threshold, the drug delivery skill 134 provides a log failed authentication response 926 comprising a notification to the user 100 after a failed validation attempt, the response 926 optionally also including direction to the user 100 to reattempt authentication 928.

### K. Illustrative Use Cases-Controlling the Drug Delivery Device

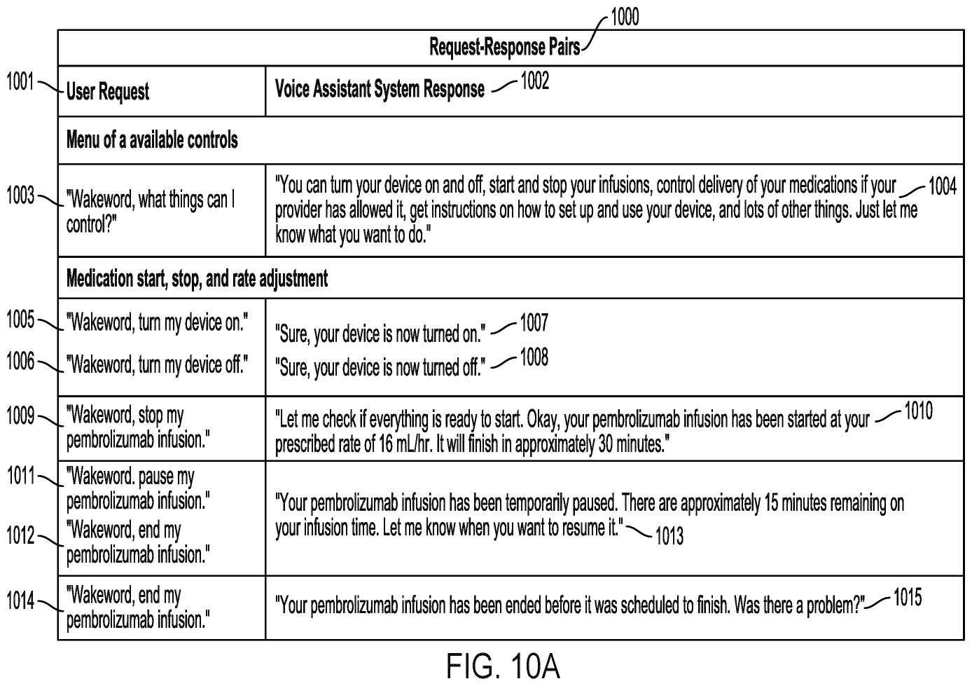

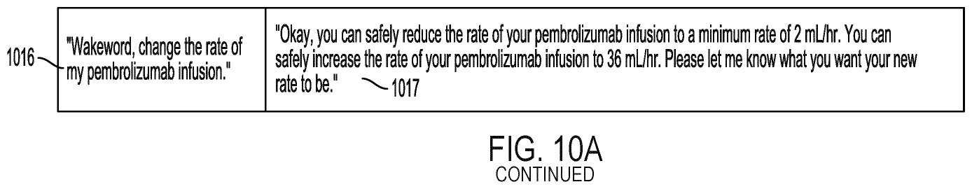

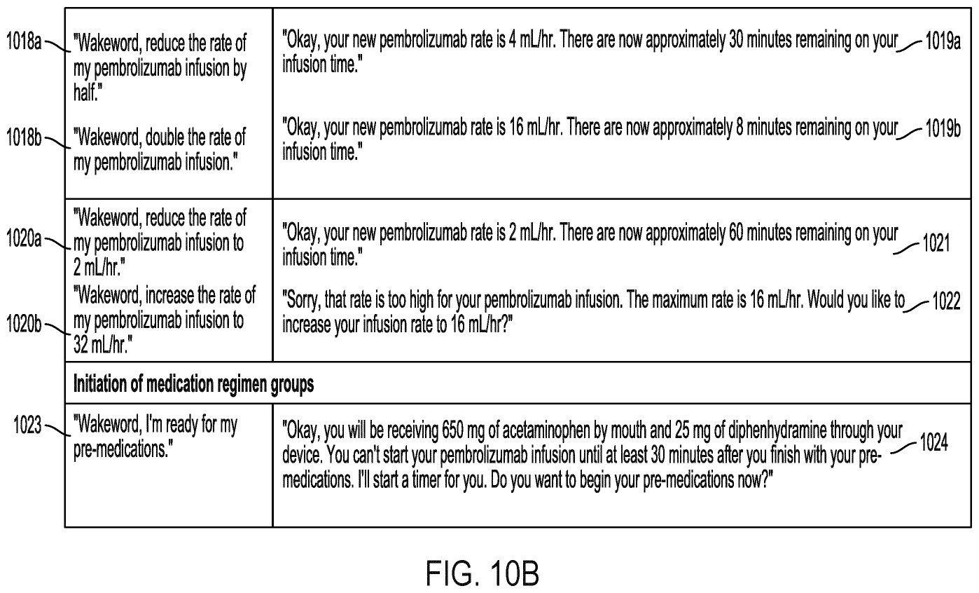

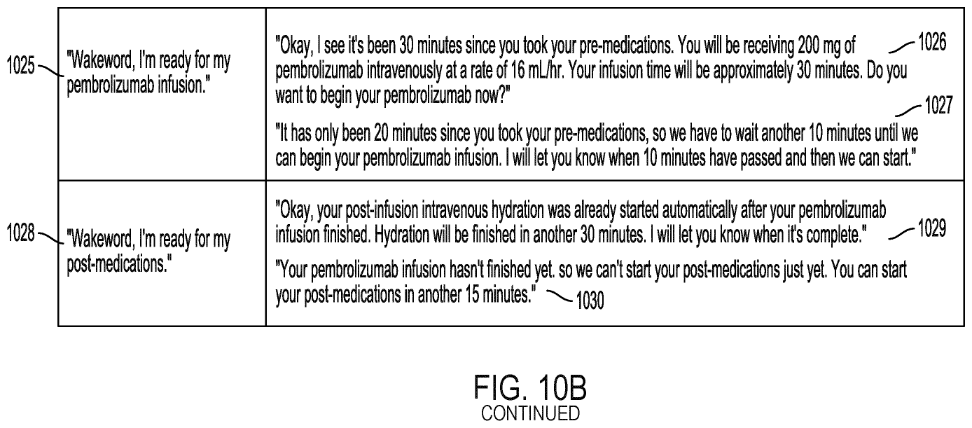

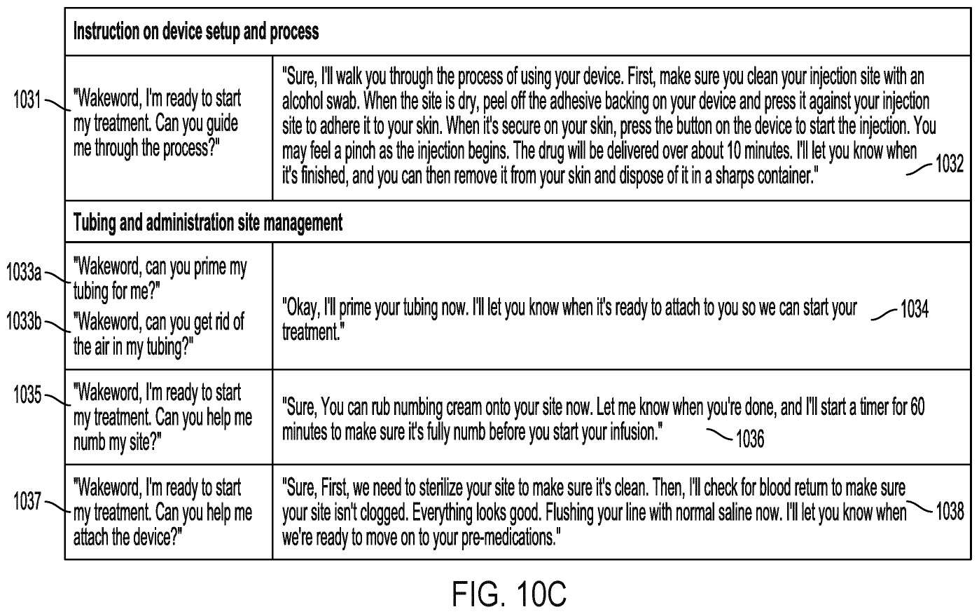

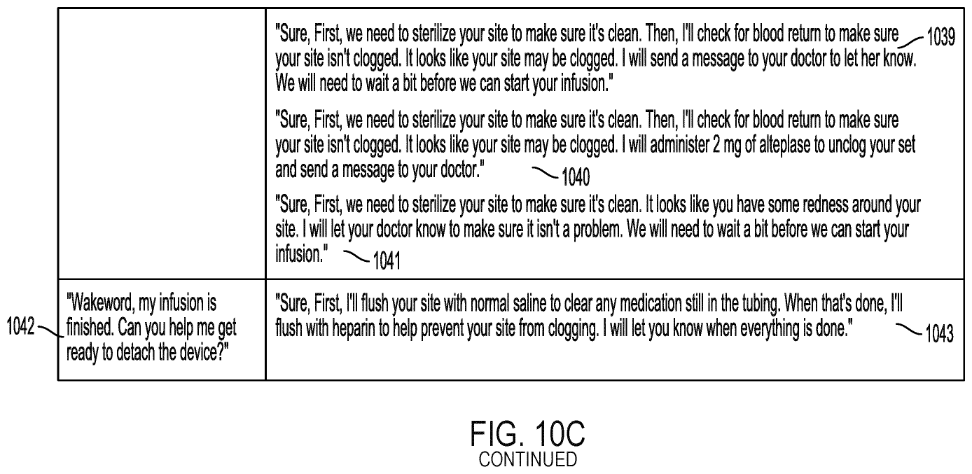

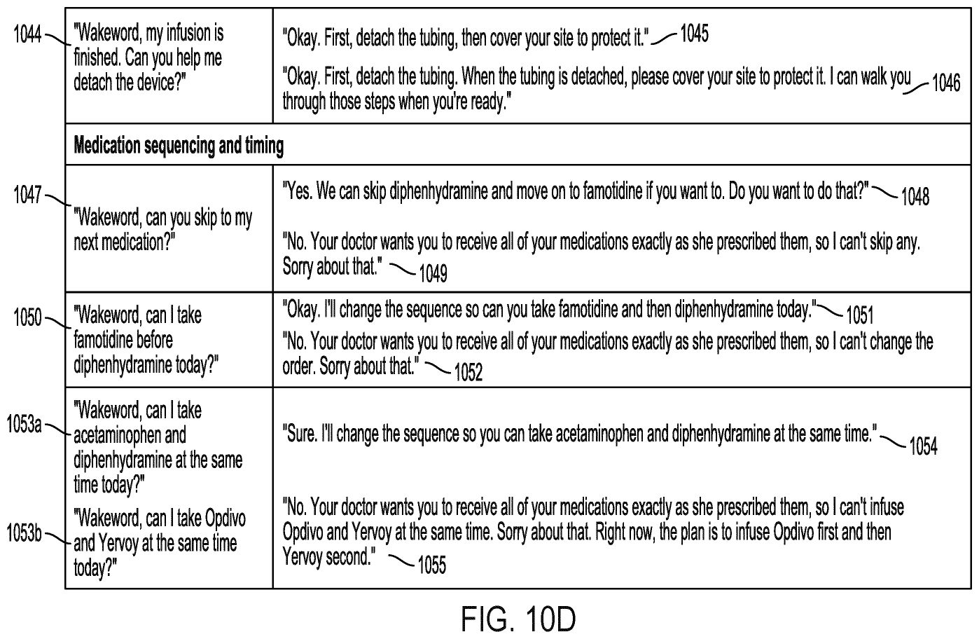

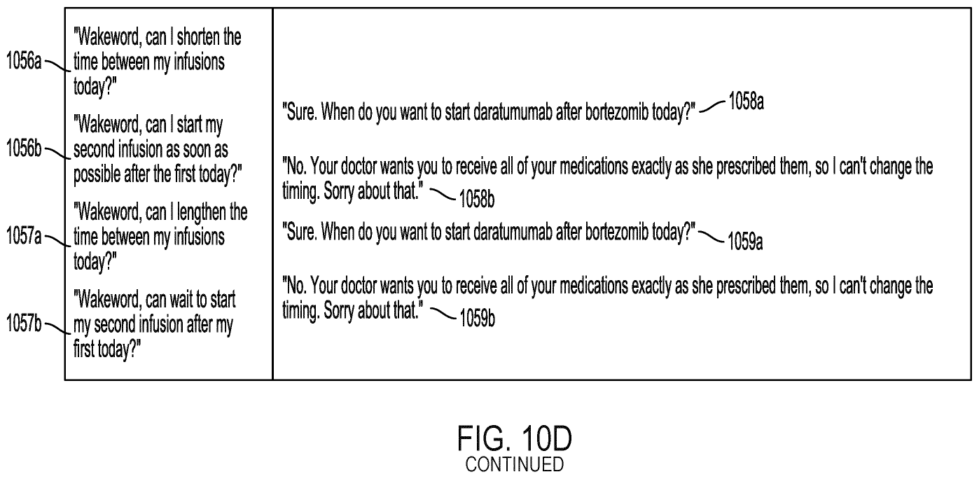

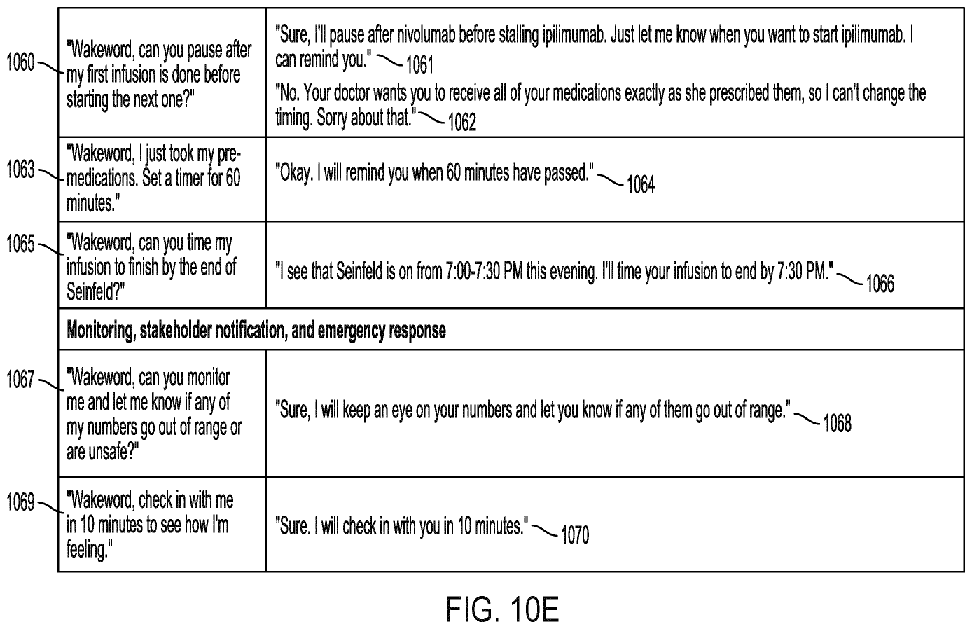

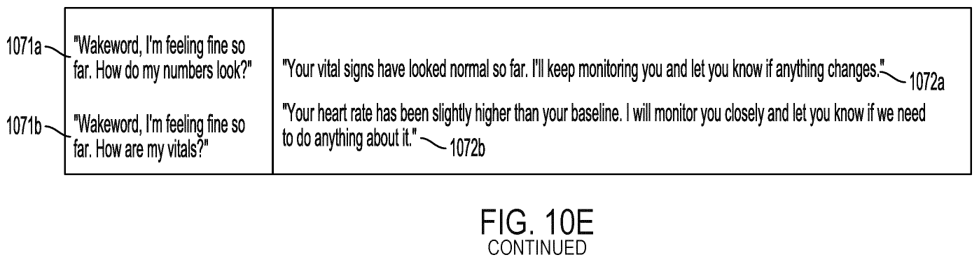

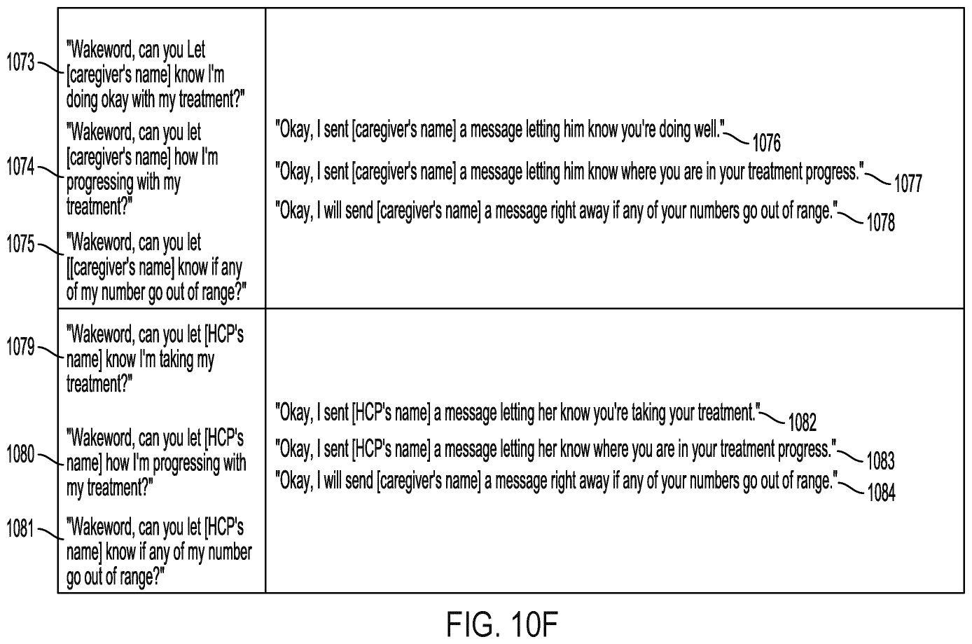

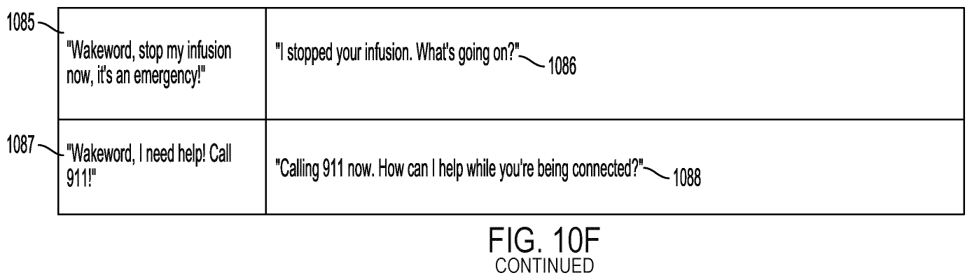

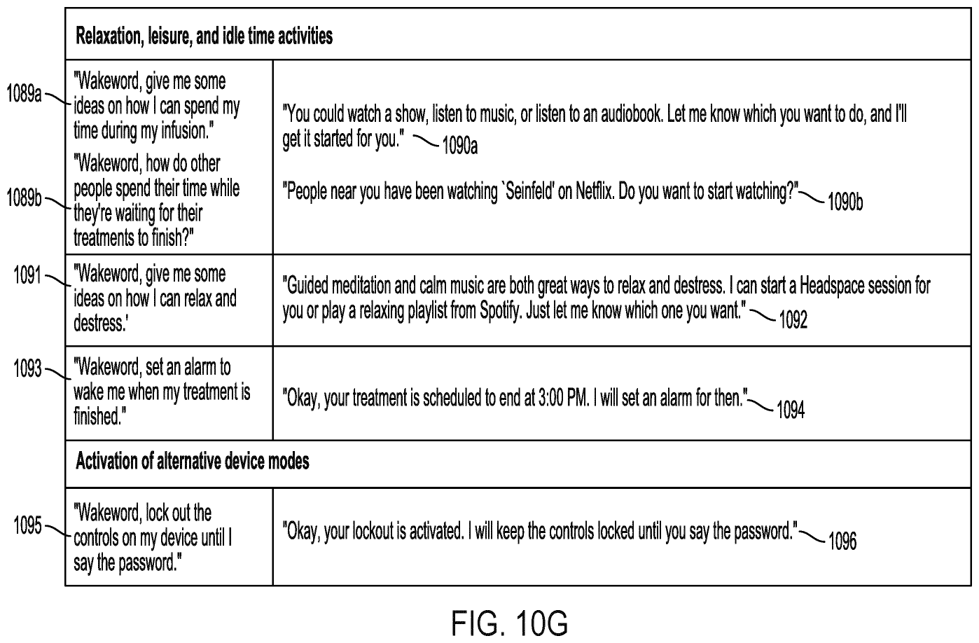

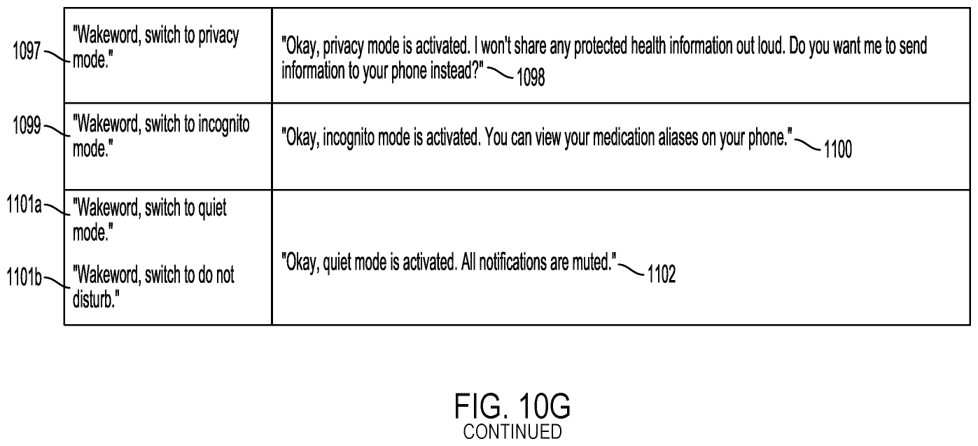

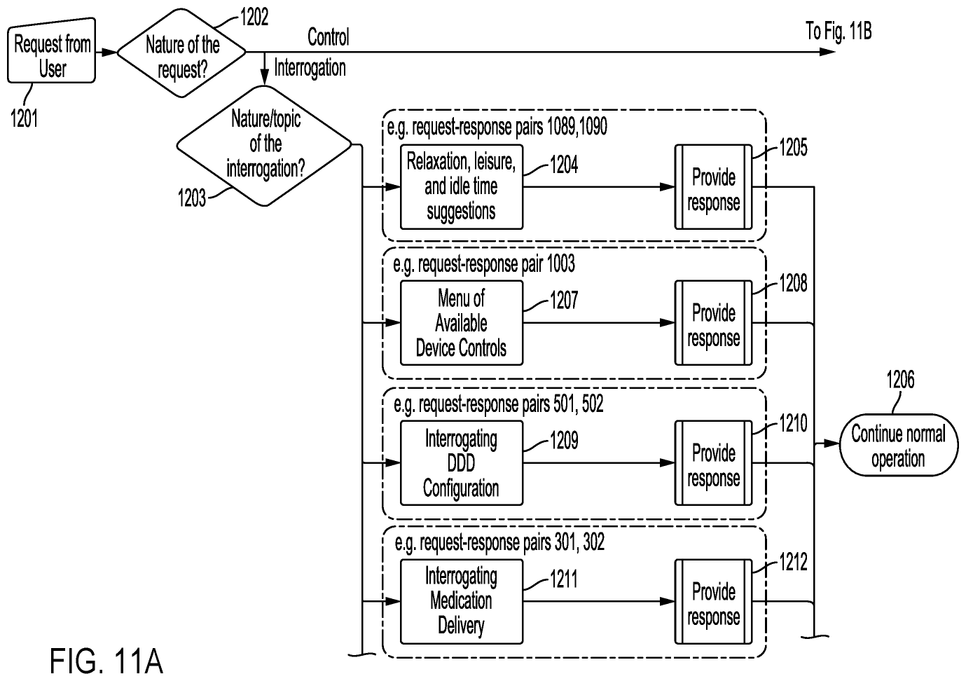

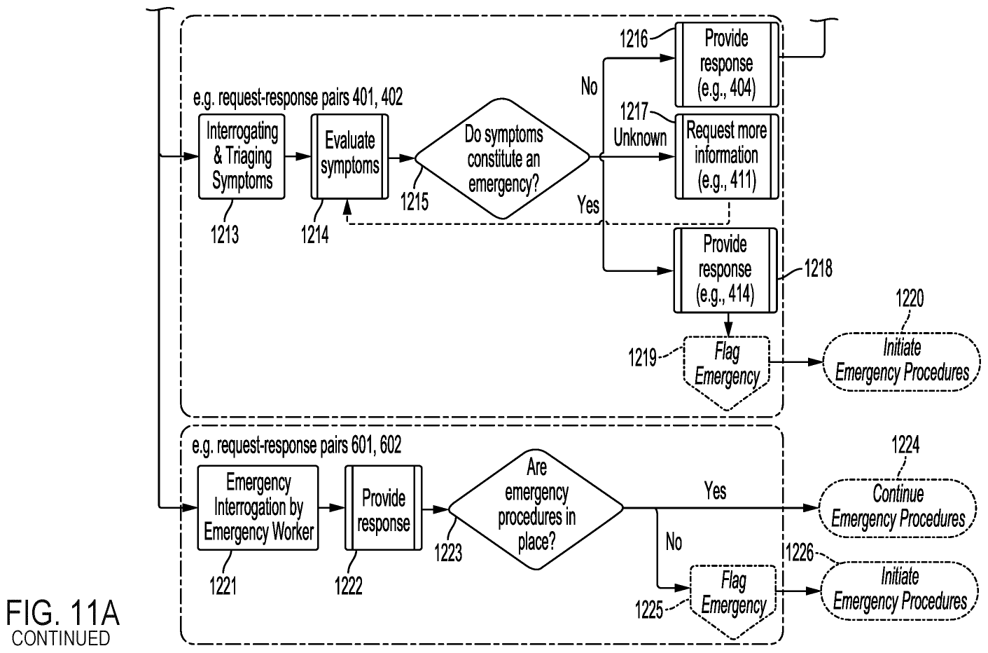

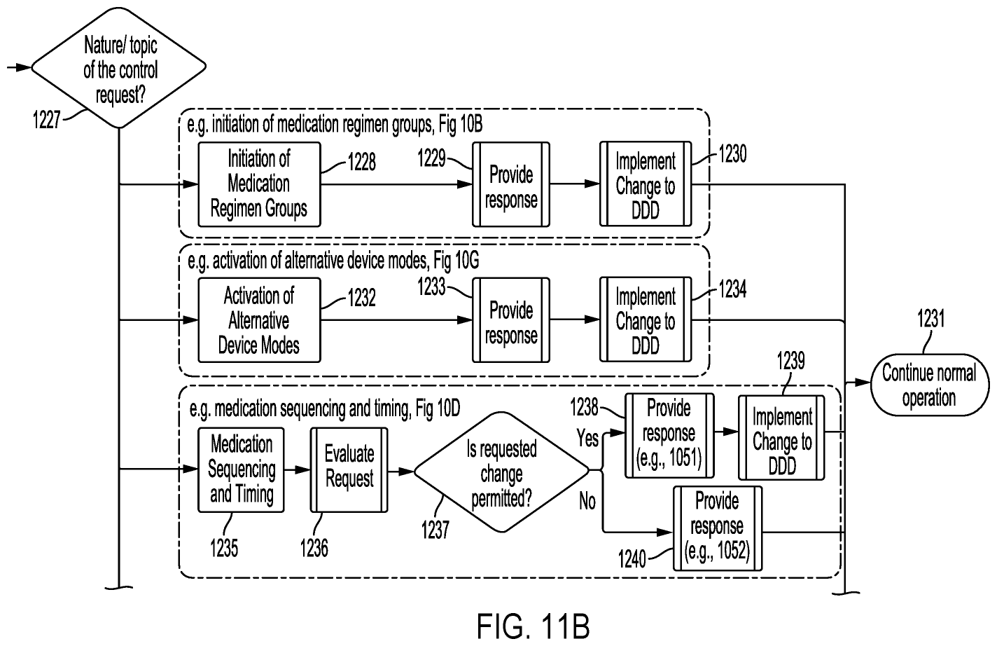

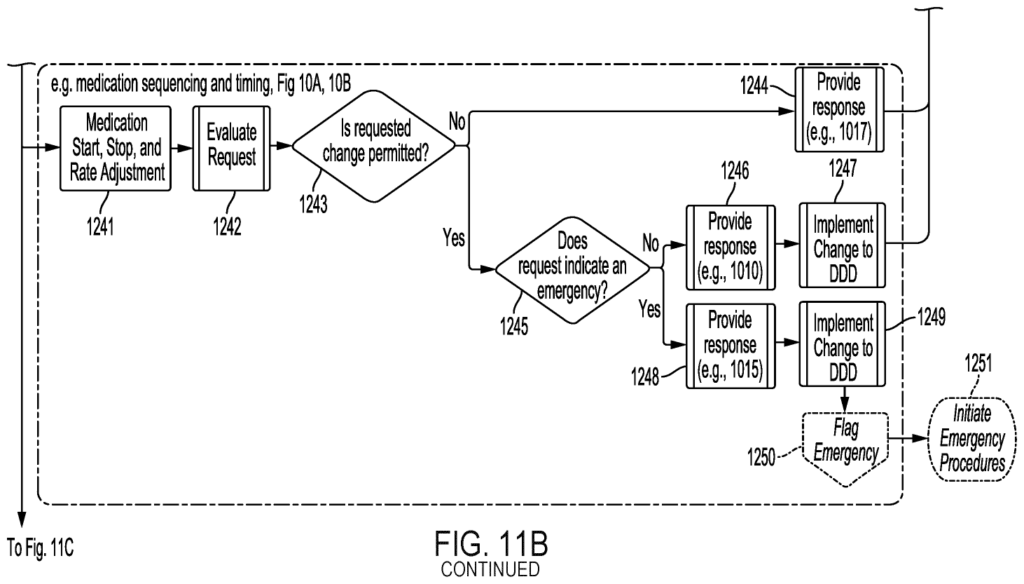

In some embodiments, the apparatus described herein is configured to control defined aspects of a drug delivery device. FIGS. 10A-10G shows a series of exemplary illustrative pairs 1000 of requests 1001 and corresponding responses 1002 related to drug delivery device control in accordance with the embodiments described herein.

In some embodiments, a user may interrogate the voice-enabled assistant for an overview of functions that can be controlled by the voice assistant 1003. Exemplary verbalized responses may include a summary of delivery device-related functions that the apparatus is configured to control 1004.

In some embodiments, a user may interrogate the voice-enabled assistant to turn the drug delivery device on 1005 and off 1006. The voice assistant response may comprise an instruction to the drug delivery device to turn the device on or off and a verbalized response including confirmation that the device was turned on 1007 or off 1008.

In some embodiments, a user may interrogate the voice-enabled assistant to start a medication infusion 1009. The voice assistant response may comprise an instruction to the drug delivery device to start a medication infusion and a verbalized response including confirmation that the medication infusion is appropriate to begin, confirmation that the medication infusion has begun at a specific rate according to a volume per unit time (e.g., mL/hr, mL/min, L/min, L/hr), mass per unit time (e.g., mg/min, mg/hr, mcg/min, mcg/hr), or volume or mass per unit weight per unit time (e.g., mcg/kg/min, mL/kg/hr), and announcement of the infusion duration 1010.

In some embodiments, a user may interrogate the voice-enabled assistant to temporarily stop 1011 or pause 1012 a medication infusion. The voice assistant response may comprise an instruction to the drug delivery device to stop or pause the medication infusion and a verbalized response including confirmation that the medication infusion has been temporarily stopped or paused, announcement of the remaining infusion duration, and clarifying prompts to resume the medication infusion if desired by the patient 1013.

In some embodiments, a user may interrogate the voice-enabled assistant to permanently end a medication infusion 1014. The voice assistant response may comprise an instruction to the drug delivery device to permanently end a medication infusion and a verbalized response including confirmation that the medication infusion has been permanently ended, that it was ended prior to when it was scheduled to finish, and clarifying prompts about a change in the patient's status that may have triggered ending the infusion 1015.

In some embodiments, a user may interrogate the voice-enabled assistant to reduce or increase the administration rate of a medication infusion without specifying the change 1016. The voice assistant response may comprise a verbalized response including the minimum or maximum administration rate that is safe or otherwise appropriate for the specific medication being delivered and clarifying prompts about the desired new rate 1017.

In some embodiments, a user may interrogate the voice-enabled assistant to reduce or increase a medication infusion rate by a proportion of the current rate 1018b, 108b. The voice assistant response may comprise an instruction to the drug delivery device to reduce or increase the administration rate of a medication infusion by the desired proportion and a verbalized response including confirmation that the medication infusion has been reduced or increased by the desired rate, announcement of the new rate, and announcement of the new infusion duration 1019a, 1019b.

In some embodiments, a user may interrogate the voice-enabled assistant to reduce or increase a medication infusion rate to a specific rate according to a volume per unit time (e.g., mL/hr, mL/min, L/min, L/hr), mass per unit time (e.g., mg/min, mg/hr, mcg/min, mcg/hr), or volume or mass per unit weight per unit time (e.g., mcg/kg/min, mL/kg/hr) 1020a, 1020b. The voice assistant response may comprise an instruction to the drug delivery device to reduce or increase a medication infusion rate to the desired rate if the rate falls within a predefined range for safety or efficacy or refusal to do so if the rate does not fall within a predefined range for safety or efficacy, and a verbalized response including confirmation that the medication infusion rate has or has not been reduced or increased to the desired rate, announcement of the new infusion rate, announcement of the new infusion duration, and clarifying prompts suggesting infusion rates based on predetermined safety and efficacy parameters 1021, 1022.

In some embodiments, a user may interrogate the voice-enabled assistant to initiate a pre-medication component of a medication regimen 1023. The voice assistant response may comprise an instruction to the drug delivery device to allow pre-medications to be administered and setting a timer to guide the user through the sequential components of the medication regimen, and a verbalized response including confirmation of which pre-medications, including drug name, dose, and route, will be administered, the amount of time required between administration of pre-medications and administration of therapeutic medications, and clarifying prompts to initiate pre-medication administration or when pre-medication administration is complete 1024.

In some embodiments, a user may interrogate the voice-enabled assistant to initiate a therapeutic medication component of a medication regimen 1025. The voice assistant response may comprise an instruction to the drug delivery device to allow or not allow therapeutic medications to be administered based on appropriate timing of other components of the regimen, and a verbalized response including confirmation of which therapeutic medications will be administered, including drug name, dose, route, and rate, announcement of the infusion duration, and clarifying prompts to initiate therapeutic medication administration or when therapeutic medication administration is complete 1026, 1027.

In some embodiments, a user may interrogate the voice-enabled assistant to initiate a post-medication component of a medication regimen 1028. The voice assistant response may comprise an instruction to the drug delivery device to allow or not allow post-medications to be administered based on appropriate timing of other components of the regimen, and a verbalized response including confirmation of which post-medications will be administered, including drug name, dose, route, and rate, announcement of the infusion duration, or clarifying prompts to initiate post-medication administration or when post-medication administration is complete 1029, 1030.

In some embodiments, a user may interrogate the voice-enabled assistant to guide them through one or more aspects of the device setup or medication delivery process, including medication preparation or setup, administration site preparation, access, or management, device setup, medication administration, and medication disposal 1031. Exemplary verbalized responses may include step-by-step guidance on site preparation, device setup and attachment, device use, and device disposal 1032.

In some embodiments, a user may interrogate the voice-enabled assistant to prime drug delivery device tubing or remove air from drug delivery device tubing in preparation for administration 1033a, 1033b. The voice assistant response may comprise an instruction to the drug delivery device to prime tubing in preparation for administration and a verbalized response including confirmation that the tubing has been primed 1034.

In some embodiments, a user may interrogate the voice-enabled assistant to help the user numb an injection site with a local anesthetic, analgesic, or other numbing agent 1035. The voice assistant response may comprise instructing the user to apply a numbing agent and setting a timer to ensure adequate analgesic effect has been achieved before the site is accessed, and a verbalized response including confirmation that the timer has been set for a specified amount of time 1036.

In some embodiments, a user may interrogate the voice-enabled assistant to help the user prepare to attach the drug delivery device to an injection site for medication administration 1037. The voice assistant response may comprise an instruction to the drug delivery device to check the injection site for patency, flush with the appropriate fluid, administer thrombolytic medications to restore patency if needed, and notify appropriate parties if any issues are detected, and a verbalized response including guidance on sterilizing the injection site, notification if an issue regarding the injection site is detected, including lack of patency or deleterious condition of the skin, confirmation of whether or not the site is appropriate to begin administration 1038, 1039, 1040, 1041.

In some embodiments, a user may interrogate the voice-enabled assistant to help the user prepare to detach the drug delivery device from an injection site after medication administration is complete 1042. The voice assistant response may comprise an instruction to the drug delivery device to flush the injection site with the appropriate fluid and administer an anticoagulant, such as heparin sodium (i.e., “heparin lock”), to reduce the risk of occlusion or loss of patency, and a verbalized response including confirmation when flushing and administration of an anticoagulant has been completed 1043.

In some embodiments, a user may interrogate the voice-enabled assistant to help the user detach the drug delivery device and secure the injection site after medication administration 1044. The voice assistant response may comprise an instruction to the drug delivery device to detach from the injection site and retain a protective barrier on the injection site, and a verbalized response including confirmation of drug delivery device detachment and guidance on how to properly secure and protect an injection site. 1045, 1046.

In some embodiments, a user may interrogate the voice-enabled assistant to skip administration of one medication and begin administration of the subsequent medication in a medication regimen 1047. The voice assistant response may comprise an instruction to the drug delivery device to stop administration of the current medication and start administration of the subsequent medication in the medication regimen, if appropriate based on predetermined order parameters, and a verbalized response including which medications would be stopped and which would be started if the skip was initiated, confirmation of whether or not the skip could be initiated, and clarifying prompts to initiate the skip 1048, 1049.

In some embodiments, a user may interrogate the voice-enabled assistant to change the sequence of one or more medications in a medication regimen 1050. The voice assistant response may comprise an instruction to the drug delivery device to change the sequence of one or medications in medication regimen, if appropriate based on predetermined order parameters, and a verbalized response including which medications would be undergo a sequence change and confirmation of whether or not the sequence change could be initiated 1051, 1052.

In some embodiments, a user may interrogate the voice-enabled assistant to administer one or medications concurrently with one or more other medications in a medication regimen 1053a, 1053b. The voice assistant response may comprise an instruction to the drug delivery device to administer one or more medications currently with one or more other medications in a medication regimen, if appropriate based on predetermined order parameters, and a verbalized response including which medications would be administered concurrently if initiated, confirmation of whether or not the concurrent administration could be initiated, and a reminder of the planned sequential administration if concurrent administration could not be initiated 1054, 1055.

In some embodiments, a user may interrogate the voice-enabled assistant to reduce or increase the time between administration of medications in a medication regimen 1056a, 1056b, 1057a, 1057b. The voice assistant response may comprise an instruction to the drug delivery device to reduce or increase the time between administration of medications in a medication regimen based on user-specified timing, if appropriate based on predetermined order parameters, and a verbalized response including confirmation of whether or not the reduction or increase in time between medication administrations could be initiated and clarifying prompts as to when the user would like to administer medications relative to other medications in a medication regimen 1058a, 1058b, 1059a, 1059b.

In some embodiments, a user may interrogate the voice-enabled assistant to pause after administration of one or more medications before initiating administration of another medication in a medication regimen 1060. The voice assistant response may comprise an instruction to the drug delivery device to pause after administration of the user-desired medication(s) and wait for confirmation before administering the next medication(s) in a medication regimen, if appropriate based on predetermined order parameters, and a verbalized response including confirmation of whether or not the pause could be initiated and clarifying prompts as to when the user would like to administer the next medication(s) in the medication regimen once resumed 1061, 1062.

In some embodiments, a user may interrogate the voice-enabled assistant to set a timer to guide sequencing of one or more medications in a medication regimen 1063. The voice assistant response may comprise setting a timer for the user-designated time and reminding the user when the time has elapsed, and a verbalized response including confirmation that the timer has been set 1064.

In some embodiments, a user may interrogate the voice-enabled assistant to time or sequence aspects of medication administration based on discrete events familiar to the user, such as the duration of a television show 1065. The voice assistant response may comprise an instruction to the drug delivery device to adjust medication administration based on the timing of discrete events familiar to the user and known by the voice assistant (e.g., calendars, television directories and schedules) and a verbalized response including confirmation of the time at which the discrete event is set to occur and confirmation that the aspect of medication delivery has been timed around that event 1066.

In some embodiments, a user may interrogate the voice-enabled assistant to monitor the user using data collected by or affiliated with the system and inform the user if any monitoring parameters are abnormal or fall outside a pre-determined range 1067. The voice assistant response may comprise an instruction to the drug delivery device to collect and/or use patient monitoring data that is already collected, and a verbalized response including confirmation that the user will be monitored and alerted if any monitoring parameters are abnormal or fall outside a pre-defined range 1068.

In some embodiments, a user may interrogate the voice-enabled assistant to check in on the user after a defined period in order to monitor the user's status during or after medication administration 1069. Exemplary verbalized responses may include confirmation that the user will be prompted for monitoring after the user-specified duration 1070.

In some embodiments, a user may interrogate the voice-enabled assistant to report patient monitoring data, including vital signs or other clinical status indicators, collected by the system 1071a, 1071b. Exemplary verbalized responses may include a patient status report based on monitoring data, confirmation that monitoring values are or are not within normal limits, and a plan for continued monitoring and any actions that will be taken if required 1072a, 1072b.

In some embodiments, a user may interrogate the voice-enabled assistant to inform a user's healthcare provider of the user's status 1079, treatment progress 1080, or if any monitoring values fall outside a pre-determined range 1081. The voice assistant response may comprise an instruction to the drug delivery device to send a message to the user-specified healthcare provider informing the healthcare provider of the user's status, treatment progress, or monitoring parameter anomalies, and a verbalized response including confirmation that such messages have been sent 1082, 1083, 1084.

In some embodiments, a user may interrogate the voice-enabled assistant to immediately stop performing an action, such as a medication infusion, due to an emergency 1085. The voice assistant response may comprise an instruction to the drug delivery device to immediately stop performing the user-specified action, and a verbalized response including confirmation that the action has been stopped and a query to the user about the emergency 1086.

In some embodiments, a user may interrogate the voice-enabled assistant to contact emergency services (e.g., dial 911) in the event of an emergency 1087. The voice assistant response may comprise an instruction to the drug delivery device to contact emergency services, and a verbalized response including confirmation that emergency services have been contacted and a query to the user about what the system can do to help in the meantime 1088.

In some embodiments, a user may interrogate the voice-enabled assistant to provide suggestions for activities the user can perform during the time required for medication administration 1089a or examples of activities other users perform during medication administration 1089b. Exemplary verbalized responses may include a list of possible activities to perform based on what's available within, interfaced with, or affiliated with the system 1090a or suggestions for particular activities based on crowdsourced data collected by the system 1090b.

In some embodiments, a user may interrogate the voice-enabled assistant to provide suggestions for relaxation or destress activities the user can perform prior to, during, or after medication administration 1091. Exemplary verbalized responses may include recommendations for relaxation or destress activities to perform based on what's available within, interfaced with, or affiliated with the system 1092.

In some embodiments, a user may interrogate the voice-enabled assistant to set an alarm to wake the user at the end of medication administration according to a medication regimen schedule 1093. The voice assistant response may comprise setting an alarm at an appropriate time based on the medication regimen schedule, and a verbalized response including confirmation that the alarm has been set for that time 1094.

In some embodiments, a user may interrogate the voice-enabled assistant to secure the device controls and only allow them to be unlocked with a spoken or entered password or passcode 1095. The voice assistant response may comprise an instruction to the drug delivery device to initiate the lockout and only allow unlocking with a spoken or entered password, and a verbalized response including confirmation that the lockout has been activated 1096.

In some embodiments, a user may interrogate the voice-enabled assistant to initiate a privacy mode in which no protected health information or otherwise sensitive information is spoken out loud by the voice-enabled assistant 1097. The voice assistant response may comprise an instruction to the drug delivery device to initiate privacy mode, and a verbalized response including confirmation that privacy mode has been initiated and a query to the user to shift communications to the user's phone in place of spoken by the voice assistant if preferred by the user 1098.

In some embodiments, a user may interrogate the voice-enabled assistant to initiate an incognito mode in which the user's medications and/or medications that can be administered by the drug delivery device are aliased to non-descriptive codenames 1099. The voice assistant response may comprise an instruction to the drug delivery device to initiate the incognito mode, and a verbalized response including confirmation that the incognito mode has been activated and a reminder that the user can view medication aliases through another medium, such as the user's phone 1100.

In some embodiments, a user may interrogate the voice-enabled assistant to a quiet mode or do not disturb mode, in which all notifications from the voice-enabled assistant or drug delivery device are muted 1101a, 1101b. The voice assistant response may comprise an instruction to the drug delivery device to initiate the quiet mode or do not disturb mode, and a verbalized response including confirmation that the quiet or do not disturb mode has been activated 1102.

In one or more embodiments, all request-response pairs require authentication prior to execution by the drug delivery skill. In one or more embodiments, no request-response pairs require authentication prior to execution by the drug delivery skill. In one or more embodiments, a subset of one or more available request-response pairs require authentication prior to execution by the drug delivery skill. In one or more embodiments, a subset of one or more available request-response pairs do not require authentication prior to execution by the drug delivery skill. In one or more embodiments, the apparatus is configured to distinguish between request requests that require authentication prior to response, those requests that do not require authentication prior to response. In one or more embodiments, one or more configured request-response pairs do not require authentication prior to execution by the drug delivery skill. In one or more embodiments, request-response pairs that do not require authentication prior to execution by the drug delivery skill include one or more functions that interrogate the current status of medication delivery or condition of the drug delivery device. In one or more embodiments, request-response pairs that do not require authentication prior to execution by the drug delivery skill include one or more functions that interrogate any emergency aspect of the medication delivery or condition of the drug delivery device. In one or more embodiments, the subset of available request-response pairs requiring authentication prior to execution by the drug delivery skill include functions that stop, start, pause, or change behavior of the drug delivery device.

In one or more embodiments, the apparatus is configured to require authentication from a user prior to any request where authorization is required. In some embodiments, the apparatus is configured to require authentication from a user periodically and provide responses to requests from that same user without authentication until the authentication period has expired. In some embodiments, the apparatus is configured to require re-authentication from a user after a previous authentication period has expired.

In one or more embodiments, the apparatus is configured to require authentication based on the environment where the apparatus will be used. In one or more embodiments, the apparatus is configured to require no authentication if used in a setting with continuous clinical observation (e.g., outpatient infusion center, hospital, or home setting with home nurse present). In one or more embodiments, the apparatus is configured to require authentication based on the presence of children or domesticated animals in the environment where the apparatus may be used.

### L. Illustrative Use Cases—Parsing the User Request Based on Interrogation or Control

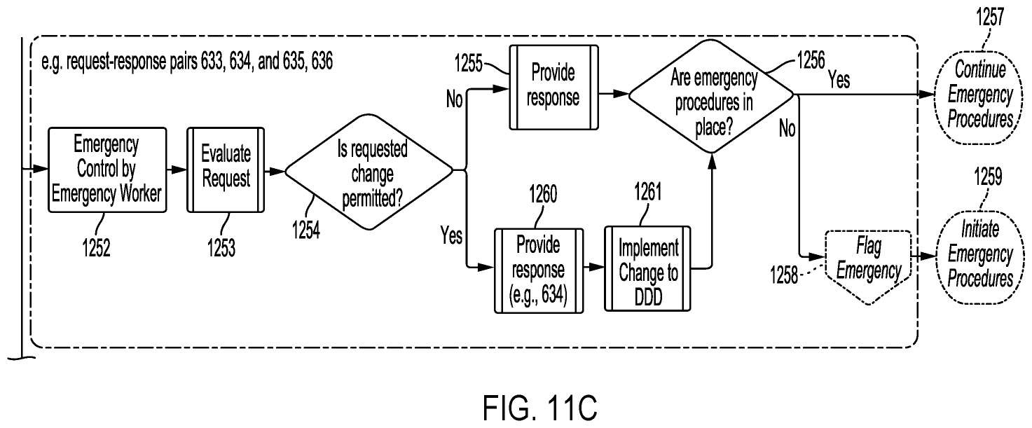

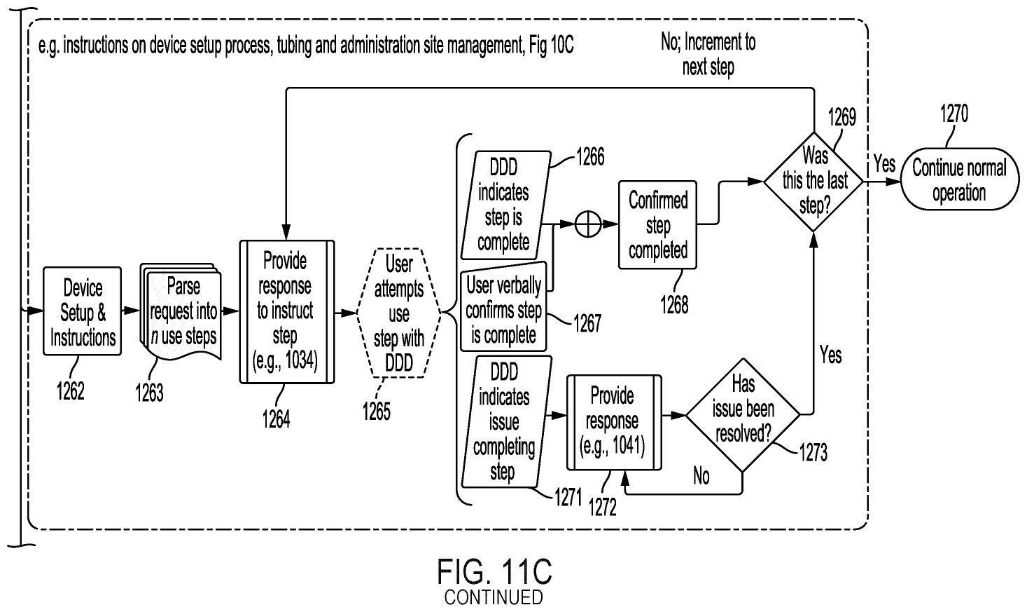

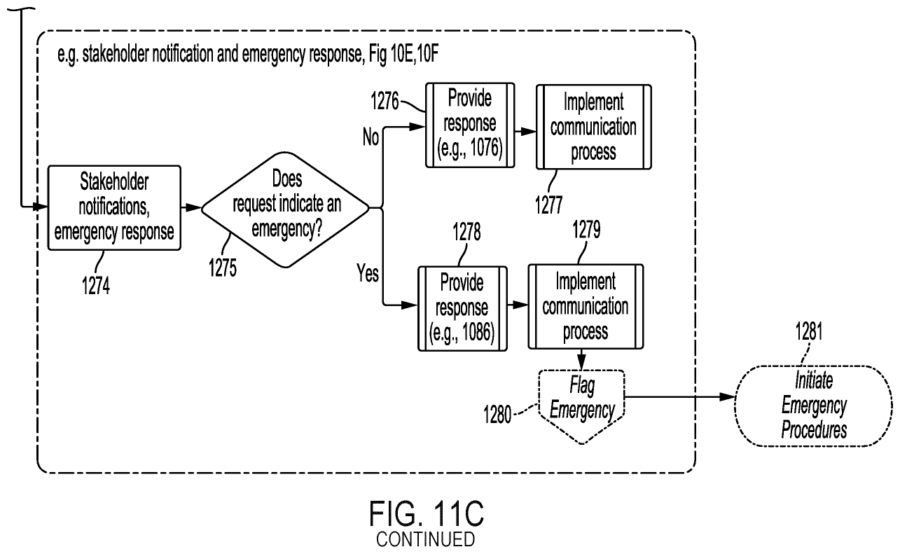

In some embodiments, the apparatus described herein is configured to control defined aspects of a drug delivery device. FIGS. 11A-11C shows a schematic drawing of a process to evaluate a request made by a user of the system, configured to distinguish between interrogation commands and control commands, according to an example embodiment.

As shown in FIG. 11A, the process begins with receiving a request from a user at block 1201. At block 1202, the nature of the request is determined. If the nature of the request is determined to be an interrogation, the process continues by determining the nature/topic of the interrogation at block 1203.

In one or more embodiments, if the nature/topic of the interrogation relates to relaxation, leisure, and idle time suggestions as shown at block 1204, the process continues by providing a response 1205 in line with the request-response pairs 1089, 1090 as non-limiting examples. The process may then continue by continuing with normal operation of the system at block 1206.

In one or more embodiments, if the nature/topic of the interrogation relates to a menu of available device controls as shown at block 1207, the process continues by providing a response 1208 in line with the request-response pair 1003 as a non-limiting example. The process may then continue by continuing with normal operation of the system at block 1206.

In one or more embodiments, if the nature/topic of the interrogation relates to interrogating drug device delivery (DDD) configuration as shown at block 1209, the process continues by providing a response 1210 in line with the request-response pairs 501, 502 as non-limiting examples. The process may then continue by continuing with normal operation of the system at block 1206.

In one or more embodiments, if the nature/topic of the interrogation relates to interrogating medication delivery as shown at block 1211, the process continues by providing a response 1212 in line with the request-response pairs 301, 302 as non-limiting examples. The process may then continue by continuing with normal operation of the system at block 1206.

In one or more embodiments, if the nature/topic of the interrogation relates to interrogating and triaging symptoms as shown at block 1213 (e.g., request-response pairs 401, 402 as non-limiting examples), the process continues by evaluating the symptoms at block 1214. If the symptoms do not constitute an emergency at block 1215, then the process continues by providing a response 1216 in line with request-response pair 404 as a non-limiting example. The process may then continue by continuing with normal operation of the system at block 1206. If it is unknown whether the symptoms constitute an emergency at block 1215, then the process continues by requesting more information at block 1217 in line with the request-response pair 411 as a non-limiting example. If the symptoms constitute an emergency at block 1215, then the process continues by providing a response 2118 in line with request-response pair 414 as a non-limiting example. The process then continues by flagging an emergency at block 1219, and initiating emergency procedures at block 1220.

In one or more embodiments, if the nature/topic of the interrogation relates to an emergency interrogation by an emergency worker as shown at block 1221 (e.g., request-response pairs 601, 602 as non-limiting examples), the process continues by providing a response at block 1222. If it is determined that emergency procedures are in place at block 1223, the process continues by continuing with emergency procedures at block 1224. If it is determined that emergency procedures are not in place at block 1223, the process continues by flagging an emergency at block 1225, and initiating emergency procedures at block 1226.

With reference to FIG. 11B, if the nature of the request is determined to be a control request, the process continues by determining the nature/topic of the control request at block 1227. In one or more embodiments, if the nature/topic of the control request relates to initiation of medication regimen groups as shown at block 1228 (e.g., the steps outlined in FIG. 10B above), the process continues by providing a response at block 1229 and implementing a change to the drug delivery device (DDD) at block 1230. The process may then continue by continuing with normal operation of the system at block 1231.

In one or more embodiments, if the nature/topic of the control request relates to activation or alternative device modes as shown at block 1232 (e.g., the steps outlined in FIG. 10G above), the process continues by providing a response at block 1233 and implementing a change to the drug delivery device (DDD) at block 1234. The process may then continue by continuing with normal operation of the system at block 1231.

In one or more embodiments, if the nature/topic of the control request relates to mediation sequencing and timing as shown at block 1235 (e.g., the steps outlined in FIG. 10D above), the process continues by evaluating the request at block 1236. If a determination that the requested change is permitted at block 1237, the process continues at block 1238 by providing a response (e.g., request-response pair 1051 as a non-limiting example) and implementing a change to the drug delivery device (DDD) at block 1239. The process may then continue by continuing with normal operation of the system at block 1231. If a determination that the requested change is not permitted at block 1237, the process continues at block 1240 by providing a response (e.g., request-response pair 1052 as a non-limiting example). The process may then continue by continuing with normal operation of the system at block 1231.

In one or more embodiments, if the nature/topic of the control request relates to mediation start, stop, and rate adjustment as shown at block 1241 (e.g., the steps outlined in FIGS. 10A-10B above), the process continues by evaluating the request at block 1242. If a determination that the requested change is not permitted at block 1243, the process continues at block 1244 by providing a response (e.g., request-response pair 1017 as a non-limiting example). The process may then continue by continuing with normal operation of the system at block 1231. If a determination that the requested change is permitted at block 1243, the process continues at block 1245 determining whether the request indicates an emergency. If a determination is made that the request does not indicate an emergency at block 1245, the process continues at block 1246 by providing a response (e.g., request-response pair 1010 as a non-limiting example) and implementing a change to the drug delivery device (DDD) at block 1247. The process may then continue by continuing with normal operation of the system at block 1231. If a determination is made that the request does indicate an emergency at block 1245, the process continues at block 1248 by providing a response (e.g., request-response pair 1015 as a non-limiting example) and implementing a change to the drug delivery device (DDD) at block 1249. The process then continues by flagging an emergency at block 1250, and initiating emergency procedures at block 1251.

With reference to FIG. 11C, in one or more embodiments, if the nature/topic of the control request relates to emergency control by an emergency worker at block 1252 (e.g., the request-response pairs 633, 634 and 635, 636 as non-limiting examples), the process continues by evaluating the request at block 1253. If a determination that the requested change is not permitted at block 1254, the process continues at block 1255 with providing a response. Next, the process continues at block 1256 by determining whether emergency procedures are in place. If it is determined that emergency procedures are in place at block 1256, the process continues by continuing with emergency procedures at block 1257. If it is determined that emergency procedures are not in place at block 1256, the process continues by flagging an emergency at block 1258, and initiating emergency procedures at block 1259. If a determination that the requested change is permitted at block 1254, the process continues at block 1260 with providing a response (e.g., request-response pair 634 as a non-limiting example) and implementing a change to the drug delivery device (DDD) at block 1261. Next, the process continues at block 1256 as outlined above.

In one or more embodiments, if the nature/topic of the control request relates to device setup and instructions at block 1262, the process continues with parsing the request into n user steps at block 1263. Next, the process continues at block 1264 with providing a response to instruction step (e.g., request-response pair 1034 as a non-limiting example). Next, at block 1265 the user attempts the use step with the drug delivery device (DDD). In one example, the process continues after block 1265 with the drug delivery device (DDD) indicating the step is complete at block 1266. In another example, the process continues after block 1265 with the user verbally confirming the step is complete at block 1267. The process then continues by confirming that the step is completed at block 1268. If it is determined that this was the last step at block 1269, the process may then continue by continuing with normal operation of the system at block 1270. If it is determined that this was not the last step at block 1269, the process may then revert back to block 1264 to provide another response to instruction step. In another example, the process continues after block 1265 with the drug delivery device (DDD) indicating that there is an issue in completing the step at block 1271. The process may then continue by providing a response at block 1272 (e.g., request-response pair 1041 as a non-limiting example). If the issue has not been resolved at block 1273, the process reverts back to block 1272 to provide another response. If the issue has been resolved at block 1273, the process continues to block 1269 as outlined above.

In one or more embodiments, if the nature/topic of the control request relates to stakeholder notifications and emergency response at block 1274. If the request does not indicate an emergency at block 1275, then the process continues by providing a response 1276 (e.g., request-response pair 1076 as a non-limiting example) and implementing a communication process at block 1277. If the request indicates an emergency at block 1275, then the process continues by providing a response 1278 (e.g., request-response pair 1086 as a non-limiting example) and implementing a communication process at block 1279. The process then continues by flagging an emergency at block 1280, and initiating emergency procedures at block 1281.

It will be appreciated that other arrangements are possible as well, including some arrangements that involve more or fewer steps than those described above, or steps in a different order than those described above.

The delivery devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjögren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behçet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the delivery devices described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the delivery devices described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the delivery devices described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, CI esterase modulators, bradykinin modulators, C-C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin gene-related peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, B-lymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation w137 (CDw137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDw123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies.

Exemplary drugs that could be included in the delivery devices described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-la, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the delivery devices described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the delivery devices described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the delivery devices described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the delivery devices described herein also include, but are not limited to, analgesics (e.g., acetaminophen), antipyretics, corticosteroids (e.g. hydrocortisone, dexamethasone, or methylprednisolone), antihistamines (e.g., diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g. an animal-derived, human-derived, or recombinant hyaluronidase enzyme), concentration modifiers or enhancers, stabilizers, buffers, or other excipients.

Exemplary drugs that could be included in the delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21,Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOpAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

While various aspects and embodiments have been disclosed herein, other aspects and embodiments will be apparent to those skilled in the art. All embodiments within and between different aspects of the devices and methods can be combined unless the context clearly dictates otherwise. The various aspects and embodiments disclosed herein are for purposes of illustration and are not intended to be limiting, with the true scope and spirit being indicated by the claims.

Some aspects of the disclosure are summarized in the following clauses.

1. A system for providing voice activated information, the system comprising:
a microphone configured to receive voice commands from a user;
a speaker configured to transmit audio messages to the user;
a medical device associated with a patient;
a processor in communication with the microphone and further in communication with the medical device associated with the patient; and
one or more computer-readable media storing computer-readable instructions that, when executed by the processor, cause the processor to:
   receive, via the microphone, a voice command associated with the medical device;
   determine a level of access control associated with the voice command;
   if the determined level of access control associated with the voice command is a first level of access control, perform an action associated with the voice command; and
   if the determined level of access control associated with the voice command is a second level of access control, transmit, via the speaker, a request for authentication prior to performing the action associated with the voice command.

2. The system of clause 1, wherein the processor is further configured to:
if the determined level of access control associated with the voice command is a second level of access control, transmit, via the speaker, a second request for authentication, wherein the second request for authentication requires a two-factor form of authentication.

3. A system for providing voice activated information, the system comprising:
a microphone configured to receive voice commands from a user;
a speaker configured to transmit audio messages to the user;
a medical device associated with a patient;
a processor in communication with the microphone and further in communication with the medical device associated with the patient; and
one or more computer-readable media storing computer-readable instructions that, when executed by the processor, cause the processor to:
   receive, via the microphone, a voice command associated with the medical device;
   analyze the voice command to determine one or more individualized biometric aspects of the first voice command;
   comparing the one or more individualized biometric aspects of the first voice command with an approved voiceprint; and
   if the one or more individualized biometric aspects of the first voice command are consistent with an approved voiceprint, perform an action associated with the voice command.

4. The system of claim 3, wherein the processor is further configured to:
if the one or more individualized biometric aspects of the first voice command are not consistent with the approved voiceprint, sending, via the speaker, a message that the action associated with the voice command will not be performed.

1-15. (canceled)

16. A system for providing voice activated information, the system comprising:
a microphone configured to receive voice commands from a user;
a speaker configured to transmit audio messages to the user;
a medical device associated with a patient;
a processor in communication with the microphone, the medical device associated with the patient, and an electronic health record system comprising an electronic health data associated with the patient; and
one or more computer-readable media storing computer-readable instructions that, when executed by the processor, cause the processor to:
receive, via the microphone, a voice command, the voice command comprising a question regarding a symptom experienced by the patient in response to an administration of an amount of medication to the patient via the medical device; and
transmit, via the speaker, one or more instructions to the user regarding the symptom experienced by the patient based on (i) the voice command, and (ii) the electronic health record system comprising an electronic health data associated with the patient.

17. The system of claim 16, wherein the voice command is received during the administration of the amount of medication to the patient.

18. The system of claim 16, wherein the voice command is received after the administration of the amount of medication to the patient.

19. The system of claim 16, wherein the one or more instructions are further based on one or more of a stage of medication administration, a medication being administered, a prior history of symptoms, or data from one or more physiologic sensors associated with the patient.

20. The system of claim 16, wherein the one or more instructions include one or more clarifying requests to perform additional triage steps to provide further context and/or enable triaging of symptoms.

21. The system of claim 16, wherein the one or more instructions comprise one or more of inducing the medical device to stop or slow an offending medication, administering a second medication to counteract the symptoms, or summon emergency services.

22. The system of claim 16, wherein the voice command received by the microphone is in a first language, wherein the one or more instructions transmitted via the speaker are in the first language.

23. The system of claim 16, wherein the voice command received by the microphone is in a first dialect of a first language, wherein the one or more instructions transmitted via the speaker are in the first dialect of the first language.

24. The system of claim 16, wherein the processor is further configured to:
receive, via the microphone, a first voice command, the first voice command having instructions for the medical device to administer the amount of medication to the patient;
send the instructions to the medical device, wherein the medical device administers the amount of medication to the patient in accordance with the instructions;

25. The system of claim 24, wherein the processor is further configured to:
authenticate the first voice command prior to sending the instructions to the medical device.

26. The system of claim 25, wherein the processor authenticates the first voice command by comparing individualized biometric aspects of the first voice command with an approved voiceprint, or wherein the processor authenticates the first voice command by:
transmitting, via the speaker, a request for authentication;
receiving, via the microphone, a passphrase from the user; and
authenticating the user based on a confirmation that the passphrase matches a stored passphrase associated with the user.

27. The system of claim 16, wherein the one or more instructions comprise an emergency response.

28. The system of claim 27, wherein the emergency response is based on one or more physiologic parameters of the patient including one or more of a respiration rate, a heart rate, an identification of heart rhythm, a blood pressure, an oxygen saturation, or a temperature.

29. The system of claim 27, wherein the processor is further configured to:
receive, via the microphone, a third voice command, the third voice command having instructions from an emergency response professional requesting information related to the medication administered to the patient via the medical device; and
transmitting, via the speaker, the requested information.

30. The system of claim 29, wherein the transmitted information includes one or more of a type of medication administered to the patient via the medical device, an amount of medication administered to the patient, and a timestamp of when the medication was administered to the patient via the medical device.
